(19) United States
(12) Patent Application Publication
(10) Pub. No.: US 2026/0124140 A1
(43) Pub. Date: May 7, 2026

(54) NON-FOULING OR SUPER STEALTH VESICLE

(71) Applicant: KAWASAKI INSTITUTE OF INDUSTRIAL PROMOTION, Kawasaki-shi, Kanagawa (JP)

(72) Inventors: Kazunori KATAOKA, Kawasaki-shi, Kanagawa (JP); Junjie LI, Kawasaki-shi, Kanagawa (JP)

(73) Assignee: KAWASAKI INSTITUTE OF INDUSTRIAL PROMOTION, Kawasaki-shi, Kanagawa (JP)

(21) Appl. No.: 19/118,501

(22) PCT Filed: Oct. 20, 2023
(86) PCT No.: PCT/JP2023/037954

§ 371 Date: Apr. 4, 2025

(30) Foreign Application Priority Data
Oct. 21, 2022 (JP) 2022-169220

(51) Int. Cl. A61K 9/1273; A61K 9/107; A61K 38/50; A61K 39/395; A61P 35/00

(52) U.S. Cl. CPC A61K 9/1273; A61K 9/1075; A61K 38/50; A61K 39/3955; A61P 35/00; C12Y 305/01001

(57) ABSTRACT

The disclosure provides non-fouling or super stealth vesicle that can be used for a drug delivery. The non-fouling or super stealth vesicle comprises polycation and polyanion, wherein the polycation and polyanion are neutralized in charge, and crosslinked to have a crosslinking degree over a certain level, and has a half-life (T_{1/2}) over 24 hours in a blood or serum of the subject.

### TECHNICAL FIELD

The disclosure relates to non-fouling or super stealth vesicle that can be used for a drug delivery.

### BACKGROUND ART

Vesicles that can deliver a drug of interest to a specific part in a body have been developed in order to increase the efficacy of the drug and/or reduce the side-effect of the drug. A polyion complex polymersome encapsulating an enzyme has been developed to improve the blood retention of the enzyme to maximize the efficacy of the enzyme administered to a body (US2021/0346472A). US2021/0346472A discloses a polyion complex polymersome encapsulating L-asparaginase where the polyion complex polymersome are made of polycation molecules and PEGylated polyanion molecules (i.e., polyanion molecules modified with polyethylene glycol (PEG), which can improve the biocompatibility of the polymersome) and these molecules are crosslinked in order to increase the stability of the polymersome in a circulation in a body. In US2021/0346472A, the polymersome showed an improved half-life that was below 24 hours in a blood circulation.

### SUMMARY OF INVENTION

The disclosure provides non-fouling or super stealth vesicle that can be used for a drug delivery. The non-fouling or super stealth vesicle comprises polycation and polyanion, wherein the polycation and polyanion are neutralized in charge, and crosslinked to have a crosslinking degree over a certain level, and has a long half-life (T_{1/2}), for example, over 24 hours, preferably 48 hours, more preferably 72 hours, still more preferably 96 hours, in a blood or serum of the subject.

The disclosure provides a polyion complex, comprising polycation and polyanion.

In an embodiment, the polycation has a positive charge, and comprises (i) a cationic polymer or a block copolymer comprising a cationic polymer block and optionally (ii) an uncharged hydrophilic polymer block. In an embodiment, the polyanion has a negative charge, and comprises (iii) an anionic polymer or a block copolymer comprising an anionic polymer block and optionally (iv) an uncharged hydrophilic polymer block. In an embodiment, the polycation and polyanion interact with each other to form the polyion complex. In an embodiment, the positive charge and the negative charge are neutralized in the polyion complex, and the ratio of the positive charge to the negative charge ranges between 0.95 and 1.05. In an embodiment, the polycation and polyanion are stabilized by crosslinking in the polyion complex and have a crosslinking density. In an embodiment, the polyion complex has a half-life (T½) over 24 hours in a blood or serum of the subject.

In an embodiment, the polyion complex is or forms a polyion complex micelle.

In an embodiment, the polyion complex is or forms a polyion complex polymersome.

In an embodiment, the crosslinking density is over 30%.

In an embodiment, the crosslinking density is over 40%.

In an embodiment, the crosslinking density is over 30%.

In an embodiment, the crosslinking density is over 40%.

In an embodiment, the sodium content in the polyion complex is 60% or less of that in a polyion complex micelle having 3.9% crosslinking density after four-hour centrifugation in 10 mM phosphate buffer (pH7.4) containing 150 mM NaCl.

In an embodiment, the sodium content in the polyion complex is 50% or less of that in a polyion complex micelle having 3.9% crosslinking density after four-hour centrifugation in 10 mM phosphate buffer (pH7.4) containing 150 mM NaCl.

In an embodiment, the sodium content in the polyion complex is 60% or less of that in a polyion complex polymersome having 7.1% crosslinking density after four-hour centrifugation in 10 mM phosphate buffer (pH7.4) containing 150 mM NaCl.

In an embodiment, the sodium content in the polyion complex is 50% or less of that in a polyion complex polymersome having 7.1% crosslinking density after four-hour centrifugation in 10 mM phosphate buffer (pH7.4) containing 150 mM NaCl.

In an embodiment, an amount of heat generation as measured by isothermal titration calorimetry after mixing 0.04 mg/mL polyion complex micelle with 20% fetal bovine serum in phosphate-buffered saline ranges between −0.1 and −0.5.

In an embodiment, an amount of heat generation as measured by isothermal titration calorimetry after mixing 0.04 mg/mL polyion complex micelle with 20% fetal bovine serum in phosphate-buffered saline ranges between −0.1 and −0.4.

In an embodiment, an amount of heat generation as measured by isothermal titration calorimetry after mixing 0.04 mg/mL polyion complex polymersome with 20% fetal bovine serum in a phosphate-buffered saline ranges between −0.1 and −0.5.

In an embodiment, an amount of heat generation as measured by isothermal titration calorimetry after mixing 0.04 mg/mL polyion complex polymersome with 20% fetal bovine serum in a phosphate-buffered saline ranges between −0.1 and −0.4.

### BRIEF DESCRIPTION OF DRAWINGS

FIG. 1 shows the schematic diagram showing the process for obtaining a polyion complex vesicle (polymersome) with or without polyethylene glycol (PEG) coating. The PEG coating was removed from the vesicle under low pH conditions.

FIG. 2 shows the electron microscopic images of the vesicle with or without PEG coating and shows the graphs showing the remarkable blood retention property of the specific polyion complex vesicle even without PEG coating. The larger graph shows the change in the relative fluorescence intensity (%) from fluorescently-labeled PIC vesicles without PEG coating, and the smaller graph inside the larger graph is a log-scale graph. M_{xx %} means a micelle that has been subjected to crosslinking treatment between the polycations and polyanions to have xx % of crosslinking density. V_{xx %} means a vesicle that has been subjected to crosslinking treatment between the polycations and polyanions to have xx % of crosslinking density. In M_{xx %} and V_{xx %}, polycations and polyanions are completely neutralized.

FIG. 3 shows the z-average (d.nm) and Cy5/Cy3 ratio of micelles and vesicles with various crosslinking degrees. In the micelles and vesicles, the polycations were labeled with Cy5 and the polyanions were labeled with Cy3. When the distance between Cy5 and Cy3 becomes within 10 nm due to compaction of the micelles or vesicles, an excitation of Cy3 will results in Cy5 remission, and thus, if the micelles or vesicles undergo compaction, the Cy5/Cy3 ration will increase.

FIG. 4 shows the time course of the change in the Cy5/Cy3 ratios of the micelles and vesicles with various crosslinking degrees were measured in a blood stream in vivo.

FIG. 5 shows the microscopic fluorescent images of the micelles with 39.5% of crosslinking density.

FIG. 6 shows the absorption of sodium ion to micelles and vesicles with various crosslinking degrees.

FIG. 7 shows the Relaxation time (T2) of the specific carbon atoms a, b, and c in micelles and vesicles with various crosslinking degrees.

FIG. 8 shows the water-solubility of the M39.5% and V30.3%.

FIG. 9 shows the amount of proteins that interact with the micelles and vesicles with various crosslinking degree by calorimetry (upper panels) and the amount of uptake of the micelles and vesicles by macrophages (lower panels) by measuring the florescent intensity of the Cy5 taken in the macrophages.

FIG. 10 shows the rapid clearance of short-circulating micelle (M33.1%) and vesicle (V20.9%) by liver sinusoidal wall based on intravital microscopic observation. Quantification of fluorescence and ratio of CD45+F4/80+ and CD45− cell populations was done by flow cytometric analysis.

FIG. 11 shows long circulating micelle (M39.5%) appear among the sinusoids after 10 h circulation compared with long circulating vesicle (V30.3%) based on intravital microscopic observation.

FIG. 12 shows long circulating micelle (M39.5%) slowly accumulates in bile canaliculus compared with long circulating vesicle (V30.3%). Quantification of fluorescence and ratio of CD45+F4/80+ cell populations was done by flow cytometric analysis.

FIG. 13 shows an anti-tumor treatment strategy by using an asparaginase-encapsulating vesicle.

FIG. 14 shows the time course of the events in the anti-tumor treatment strategy by using an asparaginase-encapsulating vesicle. Before the treatment start, a tumor cell line, 4T1 or 231/LM2 was engrafted into mice. Mice having a tumor with 50 to 100 mm³ in volume were subjected to the anti-tumor treatment strategy by using an asparaginase-encapsulating vesicle (also referred to as “ASNase®V”). The vesicle was intravenously (i.v.) administered every five days at a daily dose of 4 U per mouse. 30 days after the treatment start for 4T1, and 60 days after the treatment start for 231/LM2, the tumor and lung were collected to be analyzed.

FIG. 15 shows the effect of the administration of the asparaginase-encapsulating vesicle on the tumor weight.

FIG. 16 shows the effect of the administration of the asparaginase-encapsulating vesicle on metastasis to the lung.

FIG. 17 shows the effect of the administration of the asparaginase-encapsulating vesicle on the number of tumor nodules.

FIG. 18 shows the size and number of metastatic foci through hematoxylin and eosin (H&E) staining.

FIG. 19 shows the effect of the administration of the asparaginase-encapsulating vesicle on the plasma asparagine level 4 hours or 96 hours after the administration.

FIG. 20 shows the effect of the administration of the asparaginase-encapsulating vesicle on the asparagine level in tumor cells.

FIG. 21 shows the amount of the asparaginase delivered into tumor cells in mice to which the asparaginase-encapsulating vesicle was administered.

FIG. 22 shows the fluorescent image of a section of a tumor tissue in mice to which the asparaginase-encapsulating vesicle was administered.

FIG. 23 shows the IVIS images of mice to which the asparaginase-encapsulating vesicle and/or an immune checkpoint inhibitor (anti-PD-1 antibody, also referred to as “aPD-1”) were administered. In the IVIS images, the tumors were labeled with luciferase and visualized by administration of its substrate, luciferin.

FIG. 24 shows the intensity of bioluminescence from luciferase-expressing tumor in mice to which the asparaginase-encapsulating vesicle and/or an immune checkpoint inhibitor (anti-PD-1 antibody) were administered. The combination of the vesicle and the anti-PD-1 antibody treatments shows the most effective anti-tumor effect.

FIG. 25 shows the abdominal images of the mice to which the asparaginase-encapsulating vesicle and/or an immune checkpoint inhibitor (anti-PD-1 antibody) were administered.

FIG. 26 shows the effect of the administration of the vesicle and/or anti PD-1 antibody.

FIG. 27 shows the survival curves of the mice to which the asparaginase-encapsulating vesicle and/or an immune checkpoint inhibitor (anti-PD-1 antibody) were administered.

FIG. 28 shows the collagen distribution in sections of tumor tissue in the mice to which the asparaginase-encapsulating vesicle were administered.

FIG. 29 shows the distribution of alpha-smooth muscle actin (α-SMA) in sections of tumor tissue in the mice to which the asparaginase-encapsulating vesicle were administered.

FIG. 30 shows the distribution of anti-PD-1 antibody in sections in tumor tissue in the mice to which the asparaginase-encapsulating vesicle were administered. The asparaginase-encapsulating vesicle improved the permeability of the tumor tissue in the mice to which the asparaginase-encapsulating vesicle were administered, and then, anti-PD-1 antibody administered to the mice permeated into the tumor tissue.

FIG. 31 shows the effect of the administration of the vesicle on Collagen 1 (Coll) expression and α-SMA expression in the tumor tissue.

FIG. 32 shows the schematic diagram showing that starvation induced by the administration of the vesicle can lead to reduced desmoplasia in tumor tissues in vivo.

FIG. 33 shows the half-life of of V_{40%} where the polycation molecules forming the vesicle are 10% more abundant than the polyanion molecules forming the vesicle.

FIG. 34 shows the half-life of of V_{40%} where the polyanion molecules forming the vesicle are 10% more abundant than the polycation molecules forming the vesicle.

### DETAILED DESCRIPTION OF INVENTION

The term “subject” as used herein refers to a mammal, including dog, cat, cow, pig, horse, donkey, sheep, and primate such as monkey, gorilla, chimpanzee, orangutan, bonobo, and human.

The term “nanoparticle” as used herein refers to a particle having a size of nano meter order, for example, 10 to 1000 nm, 10 to 900 nm, 10 to 800 nm, 10 to 700 nm, 10 to 600 nm, 10 to 500 nm, 10 to 400 nm, 10 to 300 nm, 10 to 200 nm, 10 to 100 nm, 10 to 90 nm, 10 to 80 nm, 10 to 70 nm, 10 to 60 nm, 20 to 50 nm, or 30 to 40 nm.

The term “particle” as used herein refers to a particle with or without lumen inside the particle. The particle includes, but not limited to, dendrimer, vesicle, micelle, liposome, polyion complex, polyion complex micelle (PTC micelle), and polyion complex polymersome (PICsome, or also referred to as vesicle).

The term “vesicle” as used herein refers to a micelle or a hollow fine particle. The vesicle preferably has a biocompatible shell. A vesicle such as PICsome and liposome may encapsulate an active pharmaceutical ingredient such as low-molecular weight compound, protein such as enzyme, a nucleic acid, and an antibody or an antigen-binding fragment thereof.

In the present specification, the “polyion complex” (hereinafter, also referred to as “PIC”) is an ion layer formed between a cationic block and an anionic block of two block copolymers as a result of mixing a copolymer of PEG and the anionic block and a copolymer of PEG and the cationic block in an aqueous solution so as to neutralize the charges. Polyion complexes can encapsulate a therapeutic drug that can induce a therapeutic benefit to a subject that is treated with the therapeutic drug. Examples of therapeutic drugs may include small molecule drugs, middle molecule drugs, and large molecule drugs. The bonding between PEG and each of these charged chains is aimed at preventing the polyion complex from being precipitated by aggregation and at thereby allowing the polyion complex to form a nanoparticle having a monodisperse core-shell structure having a particle size of several tens of nm. In addition, PEG shells prevent protein adsorption onto nanoparticles in vivo, thereby allowing high biocompatibility and an improved blood retention time. It has been revealed that one of the charged block copolymers does not require the PEG moiety for the polyion complex formation, and this PEG moiety may be replaced with a homopolymer, a surfactant, a nucleic acid, and/or an enzyme. Furthermore, in the polyion complex formation, at least one of the anionic polymer and the cationic polymer is copolymerized with PEG, or both of these polymers may be copolymerized with PEG. As well-known, an increased content of PEG facilitates forming a PIC micelle, while a decreased content of PEG facilitates forming PICsome. Examples of the anionic polymer or block that is often used in the preparation of the polyion complex include polyglutamic acid, polyaspartic acid, and nucleic acids (e.g., DNA, mRNA, and siRNA). Examples of the cationic polymer or block include polylysine and poly(5-aminopentylaspartic acid). In this context, the mRNA means messenger RNA that is used in protein synthesis through translation. The siRNA means double-stranded RNA (nucleic acid) that can induce RNA interference (RNAi). The siRNA is not particularly limited and is double-stranded RNA of 20 to 30 bp, preferably 21 to 23 bp, 25 bp, or 27 bp, and this double-stranded RNA has a sequence homologous to the sequence of a target gene.

In this description, the terms “cationic block” and the “cationic polymer” respectively mean a polymer block and a polymer that are cationic as a whole and obtained by polymerizing monomer units containing cationic monomers. Examples of the cationic polymer include homocationic polymers and polymers in which a homocationic polymer is linked to an uncharged hydrophilic chain. When a cationic polymer forms a block copolymer together with another polymer, the cationic polymer portion may be referred to as a cationic block. In this description, the cationic polymer is a pharmaceutically acceptable cationic polymer. In this description, the term “polyvalent cation” refers to a molecule having a plurality of groups with cationic properties within the molecule, among cationic molecules. In this description, the term “polyvalent cation” can have cationic properties as a whole molecule in blood environment. Examples of the polyvalent cation include molecules that are cationic in the blood environment such as cationic polymers and cationic dendrimers. The polyvalent cation is biocompatible. In this description, the term “dendrimer” refers to a molecule having branches in a plurality of stages from one core atom.

In this description, the term “hydrophilic block” means a polymer chain that is soluble in an aqueous medium and may be a hydrophilic polymer block. In the present invention, the uncharged hydrophilic chain is a pharmaceutically acceptable uncharged hydrophilic chain. Examples of such a hydrophilic chain include poly alkylene glycol such as polyethylene glycol (PEG) and poly oxazoline such as poly(2-ethyl-2-oxazoline). The uncharged hydrophilic chain may have polar atoms, as long as the electric charge is neutralized both locally and overall. The hydrophilic block may or may not be branched. In the case where the hydrophilic block is branched, the number of branch points can be one or more.

The term “polyion complex polymersome” (hereinafter, also referred to as “PICsome”) as used herein means a hollow fine particle formed from a polyion complex. It is known that PICsome is preferably modified at the outer surface thereof with polyethylene glycol from the viewpoint of a blood retention time.

The term “micelle” as used herein means a molecular assembly formed from a single-layer molecular membrane. Examples of the micelle include a micelle formed from an amphipathic molecule such as a surfactant, and a micelle formed from a polyion complex (PIC micelle). It is known that the micelle is preferably modified at the outer surface thereof with polyethylene glycol from the viewpoint of a blood retention time. Examples of the polyion complex polymersome used in the present invention include PICsome for drug delivery. PICsome formed from a block copolymer is known as such PICsome for drug delivery. Examples of the block copolymer constituting the PICsome include a block copolymer of a PEG block and a polycation block and homopolyanion, and a block copolymer of a PEG block and a polyanion block and homopolycation. A biodegradable block copolymer is preferably employed. Various copolymers are known as such copolymers, and any of these copolymers may be used in principle. For example, a poly(aspartic acid-tetraethylenepentamine (Asp-TEP)) block copolymer and a polyethylene glycol-poly-((5-aminopentyl)-aspartic acid) block copolymer can be used as, for example, highly biocompatible and biodegradable block copolymers.

As used herein, the term “nucleic acid” includes natural nucleic acid such as natural DNA and natural RNA, antisense oligonucleotide (ASO), modified nucleic acid such as modified DNA and modified RNA, artificial nucleic acid, and a combination of these. Examples of the modified nucleic acid include fluorescent dye-modified nucleic acid, biotinylated nucleic acid, and nucleic acid into which a cholesteryl group is introduced. In order to increase the stability, RNA may have 2′-O-methyl modification, or 2′-fluoro modification or 2′-methoxyethyl (MOE) on a base or may have a replacement of a phosphodiester bond in the nucleic acid backbone by a phosphorothioate bond. Examples of the artificial nucleic acid include a nucleic acid in which the 2′-position oxygen atom and 4′-position carbon atom are crosslinked. Examples of such artificial DNA include locked nucleic acid (LNA) which is a bridged DNA in which 2′-oxygen atom and 4′-carbon atom are bridged via a methylene, and peptide nucleic acid (PNA) in which a polymer composed of amide-linked N-(2-aminoethyl) glycine is used instead of deoxyribose or ribose as backbone. Examples of RNA include artificial RNA for gene silencing such as siRNA and shRNA, micro RNA (miRNA), non-coding RNA such as natural RNA such as mRNA. These RNAs may be modified to stabilize in vivo.

The term “anti-tumor agent” or “anti-cancer agent” refers to a drug that is administered in order to treat cancer in a subject. Cancers includes, for example, solid cancers, and blood cancers. Cancers includes, for example, metastatic cancer and non-metastatic cancer.

The term “effective amount” as used herein means an amount of an antibody that can induce a desirable effect of the antibody. The desirable effect is not limited to a therapeutic effect but include non-therapeutic effect.

The term “therapeutically effective amount” as used herein means an amount of a medicine effective for treating (prevention and therapy) of a disease or a condition. A therapeutically effective amount of a medicine can reduce the speed of worsening of a symptom of a disease or a condition, stop the worsening of the symptom, improve the symptom, cure the symptom, or suppress the onset or development of the symptom.

The term “comprise” as used herein is used in the sense of including “consist of” and “essentially consist of”. The term “comprise” means that components other than target components may be contained, and the term “consist of” means that components other than target components are not contained. In this description, the term “consist essentially of” means that components other than target components are not contained in an aspect exerting a particular function (such as an aspect in which the effects of the invention are completely lost).

The present disclosure provides a polyion complex and a composition comprising the polyion complex. The polyion complex is preferably a nonfouling polyion complex or superstealth polyion complex. The polyion complex comprises polycation and polyanion. The polycation has a positive charge, and comprises (i) a cationic polymer or a block copolymer comprising a cationic polymer block and optionally (ii) an uncharged hydrophilic polymer block. The polyanion has a negative charge, and comprises (iii) an anionic polymer or a block copolymer comprising an anionic polymer block and optionally (iv) an uncharged hydrophilic polymer block. In the polyion complex, the polycation and polyanion interact with each other to form the polyion complex. In an embodiment, the positive charge and the negative charge are neutralized in the polyion complex. In a preferable embodiment, the ratio of the positive charge to the negative charge ranges between 0.95 and 1.05, between 0.96 and 1.04, between 0.97 and 1.03, between 0.98 and 1.02, or between 0.99 and 1.01 or about 1.00. In an embodiment, the polycation and polyanion are stabilized by crosslinking in the polyion complex. The neutralization of the charges from the polycation and the polyanion is important in a non-adsorptive property of a nonfouling polyion complex or superstealth polyion complex. The stabilization is also important in a non-adsorptive property of a nonfouling polyion complex or superstealth polyion complex. Those skilled in the art would adjust the ratio of the polycation and the polyanion and the degree of stabilization in order to make the polyion complex nonfouling. The Examples below show that the neutralization of the charges and the high degree of crosslinking density are sufficient to make the polyion complex non-fouling without coupling of an uncharged hydrophilic polymer block to either or both of the polycation and the polyanion. The Examples below further show that the lyophilized polyion complex can be re-constituted with water (e.g., water for injection) to form a composition comprising the polyion complex. Thus, the composition can be provided in a lyophilized form.

The disclosure therefore provides, for example, a polyion complex, comprising polycation and polyanion,
wherein the polycation has a positive charge, and comprises (i) a cationic polymer or a block copolymer comprising a cationic polymer block and optionally (ii) an uncharged hydrophilic polymer block,
wherein the polyanion has a negative charge, and comprises (iii) an anionic polymer or a block copolymer comprising an anionic polymer block and optionally (iv) an uncharged hydrophilic polymer block,
wherein the polycation and polyanion interact with each other to form the polyion complex, wherein the positive charge and the negative charge are neutralized in the polyion complex, and the ratio of the positive charge to the negative charge ranges between 0.95 and 1.05,
wherein the polycation and polyanion are stabilized by crosslinking in the polyion complex.

In an embodiment, the polyion complex has a crosslinking density. If the ratio of the positive charge to the negative charge is about 1.00, a higher crosslinking density will provide a superior non-adsorptive property of the polyion complex. Thus, in a preferable embodiment, the crosslinking density is over a predetermined crosslinking density. In a preferable embodiment, the predetermined crosslinking density is 25% or more, more preferably 30% or more, still more preferably 35% or more, 40% or more, 50% or more, 60% or more, 70% or more, 80% or more, 90% or more, 95% or more, or about 100%. A higher crosslinking density provides a stabilized ion pair network in the polyion complex to make the polyion complex non-fouling. In a preferable embodiment of the polyion complex micelle, the predetermined crosslinking density is 25% or more, more preferably 30% or more, still more preferably 35% or more, 40% or more, 50% or more, 60% or more, 70% or more, 80% or more, 90% or more, 95% or more, or about 100%. In an embodiment of the polyion complex micelle, the predetermined crosslinking density ranges between 35% and 65%. In a preferable embodiment of the polyion complex polymersome, the predetermined crosslinking density is 25% or more, more preferably 30% or more, still more preferably 35% or more, 40% or more, 50% or more, 60% or more, 70% or more, 80% or more, 90% or more, 95% or more, or about 100%. In an embodiment of the polyion complex polymersome, the predetermined crosslinking density ranges between 35% and 93%. The crosslinking density can be determined by those skilled in the art.

In an embodiment, when the polyion complex is or forms a polyion complex polymersome, a diameter (e.g., hydrodynamic diameter) in 300 mM NaCl solution of the polyion complex is 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, or 20% or less of that of a polyion complex polymersome having 7.1% crosslinking density in 300 mM NaCl solution.

In an embodiment, when the polyion complex is or forms a polyion complex micelle, a diameter (e.g., hydrodynamic diameter) in 300 mM NaCl solution of the polyion complex is 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, or 20% or less of that of a polyion complex micelle having 3.9% crosslinking density in 300 mM NaCl solution.

In an embodiment, when the polyion complex is or forms a polyion complex polymersome, the polyion complex has been crosslinked such that the sodium content in the polyion complex is 70% or less, 65% or less, 60% or less, 55% or less, 50% or less, 45% or less, 40% or less, 35% or less, 30% or less, 25% or less, 20% or less, or 15% or less of that in a polyion complex polymersome having 7.1% crosslinking density after four-hour centrifugation (e.g., ultracentrifugation, for example, with Desktop Ultracentrifuge Optima MAX-XP (Beckman Coulter) at speeds of 120,000 rpm) in 10 mM phosphate buffer (pH7.4) containing 150 mM NaCl. The sodium content after the centrifugation will decrease when counterions decrease in the polyion complex.

In an embodiment, when the polyion complex is or forms a polyion complex micelle, the polyion complex has been crosslinked such that the sodium content in the polyion complex is 70% or less, 65% or less, 60% or less, 55% or less, 50% or less, 45% or less, 40% or less, 35% or less, or 30% or less of that in a polyion complex micelle having 3.9% crosslinking density after four-hour centrifugation in 10 mM phosphate buffer (pH7.4) containing 150 mM NaCl.

In an embodiment, relaxation time T2 (s) of the crosslinked micelle is 0.03 or less, 0.025 or less, 0.024 or less, 0.023 or less, 0.022 or less, 0.021 or less, or 0.020 or less. In an embodiment, relaxation time T2 (s) of the crosslinked PICsome is 0.03 or less, 0.025 or less, 0.024 or less, 0.023 or less, 0.022 or less, 0.021 or less, or 0.020 or less. The decrease in relaxation time T2 reflects the reduced chain flexibility and the increased chain compaction in the polyion complex, which can contribute to reduce counterions that can interact with other substances, and give the micelle a non-fouling property.

In an embodiment, the polyion complex has a non-fouling property. In a preferable embodiment, an amount of heat generation as measured by isothermal titration calorimetry after mixing 0.04 mg/mL polyion complex with 20% fetal bovine serum in phosphate-buffered saline ranges between −0.1 and −0.5, more preferably between −0.1 and −0.4. In a preferable embodiment, the polyion complex is a polyion complex micelle, and an amount of heat generation as measured by isothermal titration calorimetry after mixing 0.04 mg/mL polyion complex micelle with 20% fetal bovine serum in phosphate-buffered saline ranges between −0.1 and −0.5, more preferably between −0.1 and −0.4. In a preferable embodiment, the polyion complex is a polyion complex polymersome, and an amount of heat generation as measured by isothermal titration calorimetry after mixing 0.04 mg/mL polyion complex polymersome with 20% fetal bovine serum in phosphate-buffered saline ranges between −0.1 and −0.5, more preferably between −0.1 and −0.4, still more preferably between −0.1 and −0.3. The measurement can be performed 3 to 5 minutes (for example, 3, 4, or 5 minutes) after mixing them.

When the polycation has primary amino groups and the polyanion has carboxyl groups, the primary amino group and the carboxyl group can be crosslinked, for example, by 1-ethyl-3-[3-(dimethylamino)propyl]carbodiimide (EDC). The crosslinking density will be calculated from the ratio of the crosslinked amino groups to the total amino groups that the polycation has before the crosslinking. The crosslinking density will also be calculated from the ratio of the crosslinked carboxyl groups to the total carboxyl groups that the polycation has before the crosslinking.

In an embodiment, an uncharged hydrophilic block (for example, PEG or polyoxazoline) can have an average molecular weight, for example, of 10 kD or more, 15 kD or more, 20 kD or more, 30 kD or more, or 40 kD or more (and 80 kD or less, 70 kD or less, 60 kD or less, or 50 kD or less), preferably 20 kD or more, more preferably 30 kD or more. In an embodiment, an uncharged hydrophilic block (for example, PEG or polyoxazoline) can have an average polymerization degree of 15 or more, 20 or more, 30 or more, or 40 or more (and 80 or less, 70 or less, 60 or less, or 50 or less, for example). In an embodiment, an uncharged hydrophilic block is branchless. In an embodiment, an uncharged hydrophilic block is branched.

In an embodiment, examples of the cationic polymer or the cationic polymer portion include cationic natural amino acids and cationic non-natural amino acids, e.g., cationic natural amino acids such as histidine, tryptophan, ornithine, arginine, and lysine, and/or polymer blocks having a group, as a side chain, represented by —(NH—(CH₂)₂)ₚ—NH₂ (where p represents an integer of 1 to 5) or —NH—(CH₂)_{q}—NH₂ (where q represents an integer of 1 to 10 or 1 to 5), e.g., polymer blocks of cationic non-natural amino acids having the aforementioned cationic side chain, e.g., polymer blocks of cationic non-natural amino acids such as aspartic acid or glutamic acid having the aforementioned cationic side chain. In an embodiment, the polycation block is a polymer block having a group, as a side chain, represented by —(NH—(CH₂)₂)ₚ—NH₂ (where p represents an integer of 1 to 5) or —NH—(CH₂)_{q}—NH₂ (where q represents an integer of 1 to 10 or 1 to 5). Here, preferred examples of the cationic natural amino acids include histidine, tryptophan, ornithine, arginine, and lysine, more preferably arginine, ornithine, and lysine, further preferably ornithine and lysine, furthermore preferably lysine. In an embodiment, the cationic polymer or the cationic polymer portion can be polylysine or polyornithine.

In the polycation or the polycation block, a cationic amino acid and an amino acid having a cationic side chain may be mixed. In an embodiment, the polycation block is a cationic natural amino acid, a cationic non-natural amino acid, or a polymer of monomer units containing a cationic natural amino acid and a cationic non-natural amino acid. In an embodiment, the monomer units in the polycation block are bound via peptide bonds. In a preferred embodiment, the cationic non-natural amino acid is an amino acid having a group, as a side chain, represented by —(NH—(CH₂)₂)ₚ—NH₂ (where p represents an integer of 1 to 5) or —NH—(CH₂)_{q}—NH₂ (where q represents an integer of 1 to 10 or 1 to 5). In another embodiment, the polycation block can be a polycation block formed by polymerizing a cationic natural amino acid, and glutamic acid and aspartic acid modified with a group represented by —(NH—(CH₂)₂)ₚ—NH₂ (where p represents an integer of 1 to 5) or —NH—(CH₂)_{q}—NH₂ (where q represents an integer of 1 to 10 or 1 to 5) in any order. In an embodiment, 40%, 50%, 60%, 70%, 80%, 90%, 95%, 98%, or 100% of the monomer units in the polymer has a group, as a side chain, represented by —(NH—(CH₂)₂)ₚ—NH₂ (where p represents an integer of 1 to 5) or —NH—(CH₂)_{q}—NH₂ (where q represents an integer of 1 to 10 or 1 to 5).

In an embodiment, the anion or the anionic polymer block natural amino acids and cationic non-natural amino acids, e.g., cationic natural amino acids such as glutamate and aspartate. In an embodiment, the anion or the anionic polymer block comprises polynucleotide having an anionic charge. Polynucleotides include deoxyribonucleic acid (DNA), ribonucleic acid (RNA), and a modified nucleic acid.

In an embodiment, the polyion complex has a half-life (T_{1/2}) is 30 minutes or more, 1 hour or more, 2 hour or more, 3 hour or more, 4 hour or more, 5 hour or more, 6 hour or more, 7 hour or more, 8 hour or more, 9 hour or more, 10 hour or more, 11 hour or more, 12 hour or more, 13 hour or more, 14 hour or more, 15 hour or more, 16 hour or more, 17 hour or more, 18 hour or more, 19 hour or more, 20 hour or more, 21 hour or more, 22 hour or more, 23 hour or more, 24 hour or more, 30 hour or more, 36 hour or more, 48 hour or more, 60 hour or more, 72 hour or more, 96 hour or more, or 120 hour or more. In an embodiment, the polyion complex has a half-life (T_{1/2}) that ranges between 24 hour and 168 hour, between 36 hour and 144 hour, or between 48 hour and 120 hour.

In an embodiment, the polyion complex can be incorporated into a macrophage (e.g., CD45+F4/80+ macrophage) at an amount of 90% or less, 80% or less, 70% or less, 60% or less, 50% or less, 40% or less, 30% or less, or 20% or less of that at which the reference polyion complex having a crosslinking density of between 3% to 10%.

In an Isothermal Titration Calorimetry (ITC), the amount of substances that bind to the polyion complex can be determined. The ITC is equipped with a sample cell and a reference cell. A few microliters of a phosphate-buffered saline (PBS) containing 20% fetal bovine serum (FBS) in a titration syringe is successively titrated against a polyion complex solution in the sample cell, which is maintained at a constant temperature. As the FBS solution is titrated into the sample cell and the two substances interact, heat is generated in direct proportion to the amount of binding. As the titration proceeds, the binding sites of the polyion complex in the sample cell are saturated and the thermal signal decreases until only the heat of dilution of the FBS is observed. In an embodiment, the heat generated at the first titration is 200% or less, 190% or less, 180% or less, 170% or less, 160% or less, 150% or less, 140% or less, 130% or less, 120% or less, or 110% or less of the heat of dilution of the FBS in ITC, preferably wherein 15 or more titration is required in order to the binding is saturated.

In an embodiment, the polyion complex as a vesicle can encompass a pharmaceutically active ingredient, such as small molecules, amino acids, peptides, proteins (e.g., enzyme, metabolic enzyme that is involved in a metabolic pathway), oligo nucleotide, and polynucleotide.

In an embodiment, a protein that can be encompassed into the polyion complex is an enzyme that can decompose a nutrient required for tumor or desmoplasia.

When an enzyme is encapsulated in a PICsome and brought a substrate for the enzyme into contact with the outer surface of the PICsome, the enzyme is maintained within the PICsome; however, the substrate, which is present outside the PICsome, passes through the membrane of the PICsome and successfully reacts with the enzyme within the PICsome; and the resultant reaction product can be released outside the PICsome.

Thus, according to the present disclosure, there is provided a polyion complex polymersome encapsulating an enzyme. According to the present disclosure, the enzyme can be an enzyme acting on a substance passing through the membrane of a polyion complex polymersome as a substrate.

According to the present invention, the permeability of PEG having a molecular weight of 6 kDa through the membrane of a PICsome was low. It is also predicted that the permeability of a non-fibrous protein through the membrane of a PICsome is further low. Thus, in an embodiment of the present invention, an enzyme having a molecular weight of 5 kDa or more, 10 kDa or more, 20 kDa or more, 30 kDa or more, 40 kDa or more, 50 kDa or more, 60 kDa or more, 70 kDa or more, 80 kDa or more, 90 kDa or more, 100 kDa or more, 110 kDa or more, 120 kDa or more, 130 kDa or more or 140 kDa or more can be used. If an enzyme has a molecular weight of 5 kDa or more, the enzyme can be maintained within a PICsome. The larger the molecular weight of an enzyme, the harder the enzyme to pass through the membrane of a PICsome and the easier the enzyme is maintained within the PICsome.

According to the present invention, the substrate for an enzyme is water-soluble and is specified to have a molecular weight of less than 5 kDa, 4 kDa or less, 3 kDa or less, 2 kDa or less, 1 kDa or less, 750 Da or less, 500 Da or less, 400 Da or less, 300 Da or less or 200 Da or less. If a substrate has a molecular weight of less than 5 kDa, the solubilized substrate can pass though the membrane of a PICsome. The smaller the molecular weight of a substrate, the easier the substrate passes through the membrane of a PICsome, with the result that the substrate comes easily into contact with an enzyme, and a reaction efficiency increases.

According to the present invention, a fibrous polymer was advantageously maintained within a PICsome. A branched polymer can be more advantageously maintained within a PICsome since the permeability thereof through the membrane of PICsome is lower. Accordingly, in the present invention, a fibrous protein can be used as the enzyme; however, preferably a spherical protein can be used.

According to the present invention, the enzyme encapsulated in a PICsome is stable under physiological conditions and in the blood, compared to an unencapsulated enzyme. According to the present invention, the enzyme encapsulated in a PICsome is stable particularly in the blood, compared to an unencapsulated enzyme. Accordingly, in an embodiment of the present invention, the enzyme can be an enzyme acting on a plasma component as a substrate. By taking the constitution, an enzyme and activity thereof can be stably maintained in the blood and thus can effectively treat a substrate therefor in the blood.

In an embodiment of the present invention, L-asparaginase can be used as the enzyme. L-asparaginase (hereinafter sometimes referred to as “L-ASP”) is a protein having a molecular weight of about 141 kDa, and hydrolyzes asparagine to produce aspartic acid and NH₃. L-ASP is commercially available as an agent for treating an acute lymphocytic leukemia, for example, under a trade name of Leunase™ from Kyowa Hakko Kirin Co., Ltd. L-ASP is also used in treating mastocytoma. L-ASP can be administered through intravenous injection.

In an embodiment, the enzyme may be asparaginase, which catabolizes asparagine to generate aspartic acid and NH₃, and will provide a therapeutic effect against tumor cells, including tumor cells with desmoplasia, or an asparagine-requiring tumor cell. When asparaginase is encompassed into the polyion complex of the present disclosure, the blood asparaginase level will be maintained for a longer time than a naked asparaginase level to show a stronger anti-tumor effect against tumor cells, including tumor cells with desmoplasia (which may not be asparagine-requiring tumors), or asparagine-requiring tumors.

In an embodiment, example of tumors may include, for example, but not limited to, solid cancers and hematopoietic cancers, such as a cancer selected from the group consisting of malignant melanoma, lung cancer, lung adenocarcinoma, lung cancer, small cell lung cancer, lung squamous cell carcinoma, kidney cancer, bladder cancer, head and neck cancer, breast cancer (including triple negative breast cancer), esophagus cancer, glioblastoma, neuroblastoma, myeloma, ovarian cancer, colorectal cancer, pancreatic cancer, prostate cancer, hepatocellular carcinoma, mesothelioma, cervical cancer and stomach cancer. In a preferable embodiment, the tumor may be pancreatic cancer. In an embodiment, example of tumors may include, for example, but not limited to, metastatic cancers (e.g., metastatic breast cancer and metastatic triple negative breast cancer) and non-metastatic cancers.

In an embodiment, the polyion complex having anti-tumor effect (for example, the polyion complex encapsulating an anti-tumor drug) can be administered in combination with another anti-tumor drug, such as an immune checkpoint inhibitor.

The treatment with the present polyion complex encapsulating an enzyme that can decompose a nutrient required by tumors or desmoplasia, such as asparaginase can lead to a decrease in desmoplasia in tumor tissues in a body. Desmoplasia can make the tumor inaccessible to an anti-tumor drug, due to its barrier function. Therefore, the present polyion complex can permeabilize the tumor tissues to be more accessible for anti-tumor drugs (or anti-cancer agents), especially small molecule drugs including chemotherapeutic agents and targeted therapeutic agents, middle molecule drugs including peptides or large molecule drugs including antibodies (e.g., an antibody having ADCC activity or CDC activity, or a bispecific molecule derived from an antibody), an immune check point inhibitor (e.g., small molecules and antibodies), an antibody-drug conjugate, and the like. Thus, the present polyion complex can be administered in a combination with another anti-tumor drug. Thus, the subject to be treated with anti-cancer agent may be a subject who has a decreased desmoplasia in a tumor tissue by administration of the present polyion complex encapsulating an enzyme that can decompose a nutrient required by tumors or desmoplasia, such as asparaginase.

Examples of chemotherapeutic agents may include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine)(XELODA®; ibandronate; CPT-11; topoisomerase inhibitor RFS 2000; difluoromethylornithine (DMFO); retinoids such as retinoic acid; and pharmaceutically acceptable salts, acids and derivatives of any of the above.

In some embodiments, a chemotherapeutic agent may include alkylating agents (including monofunctional and bifunctional alkylators) such as thiotepa, CYTOXAN® cyclosphosphamide, nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; temozolomide; and pharmaceutically acceptable salts, acids and derivatives of any of the above.

In some embodiments, a chemotherapeutic agent may include anthracyclines such as daunorubicin, doxorubicin, epirubicin, idarubicin, mitoxantrone, valrubicin, and pharmaceutically acceptable salts, acids and derivatives of any of the above.

In some embodiments, a chemotherapeutic agent may include an anti-hormonal agent such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); and pharmaceutically acceptable salts, acids and derivatives of any of the above.

In some embodiments, a chemotherapeutic agent may include an aromatase inhibitor that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); and pharmaceutically acceptable salts, acids and derivatives of any of the above.

In some embodiments, a chemotherapeutic agent may include an anti-androgen such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); and pharmaceutically acceptable salts, acids and derivatives of any of the above.

In some embodiments, a chemotherapeutic agent may include a protein kinase inhibitors, lipid kinase inhibitor, or an antisense oligonucleotide, particularly those which inhibit expression of genes in signaling pathways implicated in aberrant cell proliferation, such as, for example, PKC-alpha, Ralf and H-Ras.

In some embodiments, a chemotherapeutic agent may include a ribozyme such as VEGF expression inhibitors (e.g., ANGIOZYME®) and HER2 expression inhibitors.

In some embodiments, a chemotherapeutic agent may include a cytotoxic agent or antitumor antibiotic, such as dactinomycin, actinomycin, bleomycins, plicamycin, mitomycins such as mitomycin C, and pharmaceutically acceptable salts, acids and derivatives of any of the above.

In some embodiments, a chemotherapeutic agent may include a proteasome inhibitor such as bortezomib (VELCADE®, Millennium Pharm.), epoxomicins such as carfilzomib (KYPROLIS®, Onyx Pharm.), marizomib (NPI-0052), MLN2238, CEP18770, oprozomib, and pharmaceutically acceptable salts, acids and derivatives of any of the above.

In some embodiments, a chemotherapeutic agent may include an anti-microtubule agent such as Vinca alkaloids, including vincristine, vinblastine, vindesine, and vinorelbine; taxanes, including paclitaxel and docetaxel; podophyllotoxin; and pharmaceutically acceptable salts, acids and derivatives of any of the above.

In some embodiments, a chemotherapeutic agent may include an “EGFR antagonist,” which refers to a compound that binds to or otherwise interacts directly with EGFR and prevents or reduces its signaling activity, and is alternatively referred to as an “EGFR i.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, Astra7eneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidi ne-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethyla mino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-q uinazolinamine).

In some embodiments, a chemotherapeutic agent may include a tyrosine kinase inhibitor, including the EGFR-targeted drugs noted in the preceding paragraph; small molecule HER2 tyrosine kinase inhibitor such as TAK165 available from Takeda; CP724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-111 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

In some embodiments, a chemotherapeutic agent may include a retinoid such as retinoic acid and pharmaceutically acceptable salts, acids and derivatives of any of the above.

In some embodiments, a chemotherapeutic agent may include an anti-metabolite. Examples of anti-metabolites may include folic acid analogs and antifolates such as denopterin, methotrexate, pteropterin, trimetrexate; purine analogs such as fludarabine, 6-mercaptopurine, thiamiprine, thioguanine; pyrimidine analogs such as 5-fluorouracil (5-FU), ancitabine, azacitidine, 6-azauridine, carmofur, cytarabine, dideoxyuridine, doxifluridine, enocitabine, floxuridine; nucleoside analogs; and nucleotide analogs.

In some embodiments, a chemotherapeutic agent may include a topoisomerase inhibitor. Examples of topoisomerase inhibitors may include a topoisomerase 1 inhibitor such as LURTOTECAN® and ABARELIX® rmRH; a topoisomerase II inhibitor such as doxorubicin, epirubicin, etoposide, and bleomycin; and topoisomerase inhibitor RFS 2000.

In some embodiments, a chemotherapeutic agent may include a histone deacetylase inhibitor such as vorinostat, romidepsin, belinostat, mocetinostat, valproic acid, panobinostate, and pharmaceutically acceptable salts, acids and derivatives of any of the above.

Chemotherapeutic agents may also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); antirheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigal-locatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents may also include non-steroidal anti-inflammatory drugs with analgesic, antipyretic and anti-inflammatory effects. NSAIDs include nonselective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

For example, a targeted therapeutic agent may include a B-raf inhibitor, a MEK inhibitor, a K-ras inhibitor, a c-Met inhibitor, an Alk inhibitor, a phosphatidylinositol 3-kinase inhibitor, an Akt inhibitor, an mTOR inhibitor, a dual phosphatidylinositol 3-kinase/mTOR inhibitor, and combinations thereof. As used herein, the term “inhibitor” is used in the broadest sense to encompass any small molecule, protein, or other macromolecule that interferes with a biological activity of its target.

In some embodiments, a targeted therapeutic agent may include a B-Raf inhibitor such as vemurafenib (also known as Zelboraf®), dabrafenib (also known as Tafinlar®), and erlotinib (also known as Tarceva®); a MEK inhibitor, such as an inhibitor of MEK1 (also known as MAP2K1) or MEK2 (also known as MAP2K2), cobimetinib (also known as GDC-0973 or XL-518), and trametinib (also known as Mekinist®); a K-Ras inhibitor; a c-Met inhibitor such as onartuzumab (also known as MetMAb); an Alk inhibitor such as AF802 (also known as CH5424802 or alectinib); a phosphatidylinositol 3-kinase (PI3K) inhibitor such as idelalisib (also known as GS1101 or CAL-101), BKM120, and perifosine (also known as KRX-0401); an Akt inhibitor such as GSK690693, MK2206, and GDC-0941; an mTOR inhibitor such as sirolimus (also known as rapamycin), temsirolimus (also known as CCI-779 or Torisel®), everolimus (also known as RAD001), ridaforolimus (also known as AP23573, MK-8669, or deforolimus), OSI-027, AZD8055, and INK128; and a dual phosphatidylinositol 3-kinase (PI3K)/mTOR inhibitor such as XL765, GDC-0980, BEZ235 (also known as NVP-BEZ235), BGT226, GSK2126458, PF-04691502, and PF05212384 (also known as PKI-587).

In some embodiments, the anti-cancer agent is a T cell expressing a chimeric antigen receptor. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. For example, a T cell expressing a chimeric antigen receptor may express a dominant-negative TGF beta receptor, e.g, a dominant-negative TGF beta type II receptor. Examples of a treatment using a T cell expressing a chimeric antigen receptor and a dominant-negative TGF beta receptor include the HERCREEM protocol (see, e.g., ClinicalTrials.gov Identifier NCT00889954).

In some embodiments, the anti-cancer agent is an antibody or antigen-binding fragment thereof. For example, an antibody or antigen-binding fragment thereof may include alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), the antibody drug conjugate, gemtuzumab ozogamicin (MYLOTARG®, Wyeth), and combinations thereof. Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, clivatuzumab, daclizumab, eculizumab, efalizumab, epratuzumab, erlizumab, felvizumab, fontolizumab, gemtuzumab ozogamicin, inotuzumab ozogamicin, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pertuzumab, pexelizumab, ralivizumab, ranibizumab, reslivizumab, reslizumab, resyvizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, anti-IL-12 (e.g., ABT-874/J695, Wyeth Research and Abbott Laboratories, which is a recombinant exclusively human-sequence, full-length IgG₁λ antibody genetically modified to recognize IL-12 p40 protein), anti-IL-17 (e.g., MCAF5352A or RG7624), and combinations thereof.

In some embodiments, the anti-cancer agent is an antibody or antigen-binding fragment thereof that specifically binds to a target selected from CD52, VEGF-A, EGFR, CD20, HER2, HLA-DRB, CD62L, IL-6R, amyloid beta, CD44, CanAg, CD4, TNF alpha, IL-2, CD25, complement C5, CD11a, CD22, CD18, respiratory syncytial virus F, interferon gamma, CD33, CEACAM5, IL-5, integrin alpha 4, IgE, IL-4, IL-5, CD154, FAP, CD2, MUC-1, AFP, integrin αIIbβ3, ClfA, IL6R, CD40L, EpCAM, Shiga-like toxin II, IL-12, IL-23, IL-17, and CD3. In some embodiments, an antibody or antigen-binding fragment thereof that specifically binds to IL-17 (such as anti-IL-17 as described above) may include an antibody or antigen-binding fragment thereof that specifically binds to IL-17A, IL-17B, IL-17C, IL-17D, IL-17E, IL-17F, and combinations thereof.

In some embodiments, the anti-cancer agent is an antibody-drug conjugate. For example, an antibody-drug conjugate may include mertansine or monomethyl auristatin E (MMAE), such as an anti-NaPi2b antibody-MMAE conjugate (also known as DNIB0600A or RG7599), trastuzumab emtansine (also known as T-DM1, adotrastuzumab emtansine, or KADCYLA®, Genentech), DMUC5754A, bivatuzumab mertansine or cantuzumab mertansine, and an antibody-drug conjugate targeting the endothelin B receptor (EDNBR), e.g., an antibody directed against EDNBR conjugated with MMAE. For example, an antibody-drug conjugate may also include a calicheamicin or an esperamicin (e.g., calicheamicin k or esperamicin A1), such as gemtuzumab ozogamicin (MYLOTARG®, Wyeth) or inotuzumab ozogamicin. For example, an antibody-drug conjugate may also include a radioisotope chelator, e.g., a tetraxetan, such as with tacatuzumab tetraxetan or clivatuzumab tetraxetan, or a tiuxetan, as with ibritumomab tiuxetan (ZEVALIN®, Spectrum Pharma.). The term “antibody” as it relates to an antibody-drug conjugate of the present disclosure is used in the broadest sense and specifically covers monoclonal antibodies, polyclonal antibodies, multispecific antibodies (e.g., bispecific antibodies) formed from at least two intact antibodies, and antibody fragments (e.g., a Fab fragment, scFv, minibody, diabody, scFv multimer, or bispecific antibody fragment) so long as they exhibit the desired biological activity, i.e., specific binding to an antigen and the ability to be conjugated to a drug.

In some embodiments, the anti-cancer agent is an angiogenesis inhibitor. For example, an angiogenesis inhibitor may include a VEGF antagonist, e.g., an antagonist of VEGF-A such as bevacizumab (also known as AVASTIN®, Genentech); and an angiopoietin 2 antagonist (also known as Ang2) such as MEDI3617. In some embodiments, the angiogenesis inhibitor may include an antibody.

In some embodiments, the anti-cancer agent is an antineoplastic agent. For example, an antineoplastic agent may include an agent targeting CSF-1R (also known as MCSFR or CD115) such as anti-CSF-1R (also known as IMC-CS4); an interferon, e.g., interferon alpha or interferon gamma, such as Roferon-A (also known as recombinant Interferon alpha-2a); GM-CSF (also known as recombinant human granulocyte macrophage colony stimulating factor, rhu GM-CSF, sargramostim, or Leukine®); IL-2 (also known as aldesleukin or Proleukin®); IL-12; and an antibody targeting CD20 such as obinutuzumab (also known as GA101 or Gazyva®) or rituximab.

In some embodiments, the anti-cancer agent is a cancer vaccine. For example, a cancer vaccine may include a peptide cancer vaccine, which in some embodiments is a personalized peptide vaccine. In some embodiments the peptide cancer vaccine is a multivalent long peptide vaccine, a multi-peptide vaccine, a peptide cocktail vaccine, a hybrid peptide vaccine, or a peptide-pulsed dendritic cell vaccine (see, e.g., Yamada et al., Cancer Sci, 104:14-21, 2013).

In some embodiments, the anti-cancer agent is an adjuvant. Any substance that enhances an anti-cancer immune response, such as against a cancer-related antigen, or aids in the presentation of a cancer antigen to a component of the immune system may be considered an anti-cancer adjuvant of the present disclosure.

In some embodiments, the anti-cancer agent is an agent selected from a TLR agonist, e.g., Poly-ICLC (also known as Hiltonol®), LPS, MPL, or CpG ODN; tumor necrosis factor (TNF) alpha; IL-1; HMGB1; an IL-10 antagonist; an IL-4 antagonist; an IL-13 antagonist; a treatment targeting CX3CL1; a treatment targeting CXCL9; a treatment targeting CXCL10; a treatment targeting CCL5; an LFA-1 or ICAM1 agonist; and a Selectin agonist.

It is known that tumor expresses PD-L1 to suppress the activity of immune cells expressing PD-1 and the blockade of the interaction between PD-1 and PD-L1 can reactivate a tumor immunity and be effective in oncotherapy. PD-1 and PD-L1 are classified into immune checkpoint molecules and inhibitors of immune checkpoint molecules are called immune checkpoint inhibitors.

Examples of immune checkpoint inhibitors includes, for example, but not limited to, checkpoint inhibitors that block a signal through PD-1 or checkpoint inhibitors that block a signal through CTLA-4. In a preferable embodiment, checkpoint inhibitors that block a signal through PD-1 may be an antibody that can neutralize the binding between PD-1 and PD-L1 or PD-L2. In a preferable embodiment, checkpoint inhibitors that block a signal through CTLA-4 may be an antibody that can neutralize the binding between CTLA-4 and CD80 or CD86. The antibody neutralizing the binding between PD-1 and PD-L1 includes an anti-PD-1 antibody and an anti-PD-L1 antibody that can neutralize the binding between PD-1 and PD-L1. The antibody neutralizing the binding between PD-1 and PD-L2 includes an anti-PD-1 antibody and an anti-PD-L2 antibody that can neutralize the binding between PD-1 and PD-L2. The antibody that can neutralize the binding between CTLA-4 and CD80 or CD86 includes an anti-CTLA-4 antibody neutralizing the binding between CTLA-4 and CD80 or CD86. Examples of the immune checkpoint inhibitors that can be used in the present invention include, but are not particularly limited to, an anti-PD-1 antibody such as nivolumab, pembrolizumab and pidilizumab; an anti-PD-L1 antibody such as atezolizumab, durvalumab and avelumab; an anti-CTLA-4 antibody such as ipilimumab.

The present disclosure provides a composition comprising the polyion complex as explained above. The composition is suitable for delivery of a substance of interest into a target tissue. The composition can be a pharmaceutical composition. The pharmaceutical composition is used for treating a subject in need thereof in order to obtain any clinical benefit from the composition. The pharmaceutical composition can be formulated as a parenteral formulation for a parenteral administration. The pharmaceutical composition can be administered intravenously, intraperitoneally, intratumorally, intramuscularly, intraarticularly, subcutaneously, or intracutaneously. The pharmaceutical composition may comprise a polyion complex of the present disclosure and a pharmaceutically acceptable additive, such as carriers, excipients, and diluents. The pharmaceutical composition may be in a lyophilized form. The lyophilized pharmaceutical composition can be provided together with water for re-constitution or water for injection.

The disclosure provides a method of treating a subject, comprising administering to the subject, a polyion complex of the present disclosure. The disclosure provides a method of administering a molecule to a subject, comprising administering to the subject, a polyion complex of the present disclosure that encompasses the molecule.

The disclosure provides a method of producing a polyion complex of the present disclosure. The present method may comprise:
mixing a polycation having a positive charge and a polyanion having a negative charge to form a polyion complex, wherein the amounts of the polycation and the polyanion is adjusted such that the positive charge and negative charge are neutralized,
crosslinking the polycation and the polyanion by a crosslinking reagent in the polyion complex to reduce counterion in the polyion complex,
obtaining a crosslinked polyion complex. The present method may comprise:
mixing a polycation consisting of a positive charge and a polyanion consisting of a negative charge to form a polyion complex, wherein the amounts of the polycation and the polyanion is adjusted such that the positive charge and negative charge are neutralized,
crosslinking the polycation and the polyanion by a crosslinking reagent in the polyion complex to reduce counterion in the polyion complex, obtaining a crosslinked polyion complex.

The disclosure provides, for example, inventions as follows:

### Item 1

A polyion complex, comprising polycation and polyanion,
wherein the polycation has a positive charge, and comprises (i) a cationic polymer or a block copolymer comprising a cationic polymer block and optionally (ii) an uncharged hydrophilic polymer block,
wherein the polyanion has a negative charge, and comprises (iii) an anionic polymer or a block copolymer comprising an anionic polymer block and optionally (iv) an uncharged hydrophilic polymer block,
wherein the polycation and polyanion interact with each other to form the polyion complex,
wherein the positive charge and the negative charge are neutralized in the polyion complex, and the ratio of the positive charge to the negative charge ranges between 0.95 and 1.05,
wherein the polycation and polyanion are stabilized by crosslinking in the polyion complex and have a crosslinking density, and
wherein the polyion complex has a half-life (T½) over 24 hours in a blood or serum of the subject.

### Item 2

The polyion complex of Item 1, forming a polyion complex micelle.

### Item 3

The polyion complex of Item 1, forming a polyion complex polymersome.

### Item 4

The polyion complex of Item 2, wherein the crosslinking density is over 30%.

### Item 5

The polyion complex of Item 2, wherein the crosslinking density is over 40%.

### Item 6

The polyion complex of Item 3, wherein the crosslinking density is over 30%.

### Item 7

The polyion complex of Item 3, wherein the crosslinking density is over 40%.

### Item 8

The polyion complex of Item 2, wherein the sodium content in the polyion complex is 60% or less of that in a polyion complex micelle having 3.9% crosslinking density after four-hour centrifugation in 10 mM phosphate buffer (pH7.4) containing 150 mM NaCl.

### Item 9

The polyion complex of Item 2, wherein the sodium content in the polyion complex is 50% or less of that in a polyion complex micelle having 3.9% crosslinking density after four-hour centrifugation in 10 mM phosphate buffer (pH7.4) containing 150 mM NaCl.

### Item 10

The polyion complex of Item 3, wherein the sodium content in the polyion complex is 60% or less of that in a polyion complex polymersome having 7.1% crosslinking density after four-hour centrifugation in 10 mM phosphate buffer (pH7.4) containing 150 mM NaCl.

### Item 11

The polyion complex of Item 3, wherein the sodium content in the polyion complex is 50% or less of that in a polyion complex polymersome having 7.1% crosslinking density after four-hour centrifugation in 10 mM phosphate buffer (pH7.4) containing 150 mM NaCl.

### Item 12

The polyion complex of Item 2, wherein an amount of heat generation as measured by isothermal titration calorimetry after mixing 0.04 mg/mL polyion complex micelle with 20% fetal bovine serum in phosphate-buffered saline ranges between −0.1 and −0.5.

### Item 13

The polyion complex of Item 2, wherein an amount of heat generation as measured by isothermal titration calorimetry after mixing 0.04 mg/mL polyion complex micelle with 20% fetal bovine serum in phosphate-buffered saline ranges between −0.1 and −0.4.

### Item 13

The polyion complex of Item 3, wherein an amount of heat generation as measured by isothermal titration calorimetry after mixing 0.04 mg/mL polyion complex polymersome with 20% fetal bovine serum in a phosphate-buffered saline ranges between −0.1 and −0.5.

### Item 14

The polyion complex of Item 3, wherein an amount of heat generation as measured by isothermal titration calorimetry after mixing 0.04 mg/mL polyion complex polymersome with 20% fetal bovine serum in a phosphate-buffered saline ranges between −0.1 and −0.4.

### EXAMPLES

### Example 1

### Materials and Methods

Materials. α-Methoxy-ω-amino poly(ethylene glycol) (Mₙ=2,200, 5,500 or 12,000) was purchased from NOF Co., Ltd. (Tokyo, Japan). β-Benzyl-L-aspartate N-carboxy-anhydride (BLA-NCA) was purchased from Chuo Kaseihin Co., Ltd. (Tokyo, Japan). 1,5-Pentanediamine, n-butylamine, 2,2-bis(aminoethoxy)propane, acetic anhydride, fluorescamine, 1-ethyl-3-(3-dimethylaminopropyl)carbodiimide hydrochloride and 5-carboxyfluorescein diacetate were purchased from Sigma-Aldrich. α-Methoxy-ω-4-nitrophenoxy carbonyl polyoxyethylene (M_{w}=2,000) and methoxy PEG succinimidyl carboxymethyl ester (PEG-NHS, M_{w}=5,000) were obtained from Yuka Sangyo Co. Ltd. (Tokyo, Japan). Solvents used for NCA polymerization (dichloromethane and dimethylformamide) were purified by Ultimate Solvent System 4-2S-TKII (Osaka, Japan). Asparaginase (10000 U) was provided by Kyowa Hakko Kirin Co., Ltd. (Tokyo, Japan). Sulfo-Cy3 NHS ester (Cy3-NHS) and Sulfo-Cy5 NHS ester (Cy5-NHS) were purchased from Lumiprobe Corporation (Hallandale Beach, Florida, USA). Bouin solution and 4% paraformaldehyde were purchased from Wako Pure Chemical Industries, Ltd. (Osaka, Japan). Cell lysis buffer was purchased from Promega Corporation (Madison, USA). ACK lysis buffer was purchased from Gibco by Life Technologies (New York, USA). DAPI was obtained from Thermo Scientific (Waltham, USA). Deproteinizing Sample Preparation Kit-TCA (ab204708) and Asparagine Assay Kit (ab273333) were purchased from Abcam (Cambridge, UK). FITC anti-mouse CD45 Antibody (#103108) and PE anti-mouse F4/80 Antibody (#123110) were provided by Biolegend (San Diego, USA). Anti-mouse F4/80 antibody (eFluor 450, #48480182) was purchased from eBioscience. Alexa Fluor® 488 anti-alpha smooth muscle actin antibody [1A4](ab184675), anti-collagen I antibody (ab21286), and anti-rabbit IgG H&L (Alexa Fluor® 488, abl50077) was obtained from abcam. InVivoMAb anti-mouse PD-1 (CD279, clone: RMP1-14) was purchased from Bio X Cell (Lebanon, USA).

Cell lines. The primary KPC cell line was derived from a genetically engineered mouse model (LSL-Kras^{G12D/+}, LSL-Trp53^{R172H/+}, and Ptfla-Cre) and provided by Dr. Ryo Tsumura (Division of Developmental Therapeutics, EPOC, National Cancer Center, Kashiwa, Japan). MDA-MB-231-LM2-4175 (referred to as 231/LM2) human triple negative breast cancer (TNBC) cell line with spontaneous lung metastasis following orthotropic implantation was generously provided by Prof. Joan Massague (Memorial Sloan Kettering Cancer Center). 4T1 mouse TNBC cell line and RAW264.7 cell line were purchased from Japanese Collection of Research Bioresources (JCRB) Cell Bank (Osaka, Japan). KPC, 231/LM2, and 4T1 cells were stably transfected with the lentiviral vector carrying firefly luciferase (Luc). 231/LM2 cells were also transfected with GFP. Cells were routinely cultured at 37° C. in DMEM medium (Gibco) supplemented with 10% FBS (Invitrogen), 100 U/ml penicillin (Invitrogen) and 100 U/ml streptomycin (Invitrogen) in a humidified atmosphere containing 5% CO₂.

Animals. 5-Week-old female C57BL/6J mice, BALB/c mice and BALB/c nude mice were purchased from Charles River Co. (Tokyo, Japan). The animal studies were carried out according to the ethical guidelines of the Innovation Center of NanoMedicine (Kawasaki, Japan).

Synthesis and characterization of polymers. PEG-b-poly(5-aminopentyl-aspartamide) (PEG-b-P(Asp-AP)), homo-poly(5-aminopentyl-aspartamide) (P(Asp-AP)) and PEGb-poly(aspartic acid) (PEG-b-PAsp) were prepared according to our previous report¹⁶. Typically, PEG-b-poly(β-benzyl-L-aspartate) (PEG-b-PBLA) and homopoly(β-benzyl-L-aspartate) (homo-PBLA) were first synthesized by ring-opening polymerization (ROP) of β-benzyl-L-aspartate N-carboxyanhydride (BLA-NCA) using PEG-NH₂ (Mₙ, =2,200, 5,500 or 12,000 g mol⁻¹) and n-butylamine as the initiator, respectively. Then, further hydrolysis (under alkaline conditions) and aminolysis (with 1,5-pentanediamine) of benzyl ester groups from PBLA segments yielded carboxyl-based polyanions (PEG-b-PAsp) and amino-based polycations (PEG-b-P(Asp-AP) and homo-P(Asp-AP)), respectively, as white powder following lyophilization. Degree of polymerization (DP) was determined by ¹H NMR spectra recorded on NMR JNM-ECS400 (JEOL) spectrometer (Akishima, Japan) using DMSO-d₆ or D₂O as the solvent. The molecular weight (MWs) and molecular weight distribution (M_{w}/M_{w}) were determined by gel permeation chromatography (GPC) equipped with a superdex 200 10/300 column (GE Healthcare, Little Chalfont, UK). The eluent was 10 mM phosphate buffer (pH 7.4) containing 500 mM NaCl for polyanions and 10 mM aqueous acetic acid solution containing 500 mM NaCl for polycations at a flow rate of 0.5 mL min⁻¹. For fluorescence studies, the w-amino group of PBLA was used to label sulfo-Cy3 or sulfo-Cy5. Identically, further hydrolysis or aminolysis afforded homoP(Asp-AP)-Cy5 and PEG-b-P(Asp-AP)-Cy5 as blue powder and PEG-b-PAsp-Cy3 as a purple powder following lyophilization.

Typical synthetic procedures for preparation of PEG-removable polyanion, PEG-ketal-PAsp with pH-responsive ketal linker, were as follows. Briefly, 2,2-bis(aminoethoxy)propane (0.81 g, 5 mmol) was dissolved in dichloromethane (30 mL), followed by followed by slow dropwise addition of α-methoxy-ω-4-nitrophenoxy carbonyl polyoxyethylene (PEG-pNP, M_{w}2,000, 1 g, 0.5 mmol) in dichloromethane (30 mL). The reaction continued at 25° C. for 6 h and the resulting solution was concentrated and precipitated into diethyl ether, obtaining PEG-ketal-NH₂. Lyophilized PEG-ketal-NH₂ (0.1 g, 0.05 mmol) from benzene was subsequently utilized to initiate the ROP of BLA-NCA (1.18 g, 4.75 mmol) in anhydrous dimethylformamide/dichloromethane (5 mL/20 mL) at 25° C. for 96 h under argon atmosphere. The reaction mixture was precipitated into diethyl ether, obtaining PEG-ketal-PBLA. Then, PEG-ketal-PBLA (185 mg, 0.01 mmol) was dissolved in chloroform (2 mL), followed addition of sodium hydroxide (32 mg) in water/methanol/2-propanol (0.2 mL/0.4 mL/0.4 mL). After vigorously stirring for 15 min at 4° C., 270 L acetic acid was added into the reaction system. Chloroform was removed by evaporation and the remaining solution was dialyzed against 10 mM PB (pH 7.4) and subsequently, deionized water at 4° C. The white powder, PEG-ketal-PAsp was obtained after lyophilization. The characterizations of all used polymers were listed in Table 1.

TABLE 1
Characterizations of polycations and polyanions used in this study

| | PEG molecular | DP of polycation | |
|---|---|---|---|
| | weight | or polyanion | |
| Polymers | (Mn) | segment^{a} | M_{w}/Mₙ^{b} |
| Homo-P(Asp-AP) | — | 81 | 1.16 |
| PEG-b-P(Asp-AP) | 2,200 | 80 | 1.17 |
| PEG-b-P(Asp-AP) | 5,500 | 71 | 1.13 |
| PEG-b-P(Asp-AP) | 12,000 | 77 | 1.15 |
| PEG-b-PAsp | 2,200 | 80 | 1.05 |
| PEG-b-PAsp | 5,500 | 75 | 1.06 |
| PEG-b-PAsp | 12,000 | 74 | 1.07 |
| PEG-ketal-PAsp | 2,200 | 82 | 1.06 |
| homo-P(Asp-AP)-Cy5 | — | 77 | 1.15 |
| PEG-b-P(Asp-AP)-Cy5 | 2,200 | 81 | 1.10 |
| PEG-b-P(Asp-AP)-Cy5 | 5,500 | 76 | 1.18 |
| PEG-b-P(Asp-AP)-Cy5 | 12,000 | 74 | 1.13 |
| PEG-b-PAsp-Cy3 | 2,200 | 78 | 1.05 |

^{a}Degree of polymerization (DP) per polymer was determined by ¹H NMR spectra.
^{b}Mw/Mn was determined by GPC.

Construction of the continuum of PIC nanoparticles. All polycations and polyanions were separately dissolved in 10 mM PB (pH 7.4) with final concentration of 1 mg mL⁻¹ as stock solutions. PEG-b-PAsp and PEG-b-P(Asp-AP) at stoichiometric charge ratio were physically mixed and vortexed for 2 min, followed by addition of varying amounts of 1-ethyl-3-(3-dimethylaminopropyl)carbodiimide hydrochloride (EDC; 0.1, 0.25, 0.5, 1, 2, 3, 4, 5, 6, 8, 10, 20, 50 equivalents to carboxyl/amino group). The crosslinking reaction was continued at 25° C. for 6 h. The obtained continuum of PIC micelles with varying crosslinking density were purified by ultrafiltration using polyethersulfone membranes (MWCO of 50 kDa). PEG-b-PAsp and homo-P(Asp-AP) were used for construction of continuum of PIC vesicles using the procedure identically as above mentioned. Similarly, PEG-ketal-PAsp and homo-P(Asp-AP) were used to prepare PEG-removable PIC vesicles. For fluorescence studies, 25 mol % Cy5-labelled polycations (PEG-b-P(Asp-AP)-Cy5 or P(Asp-AP)-Cy5) were added. To formulate PIC nanoparticles coupled with FRET pair, 25 mol % Cy5-labelled polycations (PEG-b-P(Asp-AP)-Cy5 or P(Asp-AP)-Cy5) and 25 mol % Cy3-labelled polyanions (PEG-b-PAsp-Cy3) were added. Note that for each independent construction of a continuum, all PIC nanoparticles were prepared in large quantities (e.g., 100 mL) and then distributed for further crosslinking.

Quantification of crosslinking density. Crosslinking density of the continuum was independently quantified by two methods. First, fluorescamine assay was used to detect the content of primary amines. PIC nanoparticles at 0.01, 0.005, 0.0025 and 0.00125 mg mL⁻¹ in 10 mM pH 7.4 PB containing 500 mM NaCl (75 uL) were mixed with 0.3 mg mL⁻¹ fluorescamine solution in acetone (25 μL) and incubated at 25° C. for 15 min. The fluorescence signal was monitored by microplate reader (TECAN, Infinite M1000 PRO) with a filter set of Ex/Em=390/475 nm. Linear regression analysis of fluorescence-concentration plots was applied to obtain the slope. The crosslinking density was calculated as follows. Crosslinking density (%) (1-slope/slope_{100%})×100%, where slope_{100%} represents the slope of fluorescence-concentration plot for noncrosslinked polyion complex nanoparticle. Crosslinking density was further independently confirmed through quantification of carboxyl group using attenuated total reflectance-fourier transform infrared (ATR-FTIR) spectroscopic method.

Construction of PIC vesicle without PEG coating. PEG-removable PIC vesicle with 32.6% crosslinks was treated with 10 mM hydrochloric acid solution overnight, followed by ultrafiltration using polyethersulfone membranes (MWCO of 50 kDa). Complete removal of PEG was confirmed by ¹H NMR.

Construction of therapeutic nanoreactor. Asparaginase (ASNase) was loaded into PIC vesicle to construct therapeutic nanoreactor (ASNase@V). ASNase in 10 mM pH 7.4 PB was simply added into solution in the process of mixing of homo-P(Asp-AP) and PEG-b-PAsp, followed by vortex for 2 min. Further crosslinking by 2 equivalents of EDC and purification using polyethersulfone membranes (MWCO of 300 kDa) afforded nanoreactor. Loading content was optimized by varying ASNase feeding concentration (0.1, 0.2, 0.3, 0.4, 0.5, 0.6, 0.7, 0.8, 0.9, 1, 2, 5 mg mL⁻¹) and quantified through fluorescence of Cy5-labelled ASNase. The ASNase® V with loading content of 2.91% was used for further studies.

Dynamic light scattering (DLS), Forster resonance energy transfer (FRET) and (cryo)-transmission electron microscopy (TEM/cryo-TEM) measurements. PIC nanoparticles were adjusted to 0.5 mg mL⁻¹ in 10 mM pH 7.4 PB in the absence or presence of 150 mM or 300 mM NaCl. Then, particles sizes, particle size distributions, scattering intensity and zeta-potential were measured by Malvern Zetasizer Nano ZS90 with a He—Ne laser (633 nm) and 173° collecting optics (Malvern Instruments, Worcestershire, UK). FRET spectra were recorded by microplate reader (TECAN, Infinite M1000 PRO) with excitation wavelength at 540 nm and emission wavelength range of 540-780 nm. The ratio of emission intensity at 670 nm to 566 nm (Cy5/Cy3) was quantified. Meanwhile, 3 mg mL⁻¹ PIC nanoparticles in deionized water were used to prepare TEM and cryo-TEM samples, followed by imaging on JEOL JEM-1400 transmission electron microscope (120 kV) and Tecnai G2 Spirit BioTWIN electron microscope (120 kV), respectively.

Spin-spin relaxation time (T2) measurement. PIC nanoparticles were constructed in 10 mM phosphate buffer/D₂O. The purified solutions containing 150 mM NaCl at 20 mg mL⁻¹ for PIC nanoparticles were used for NMR relaxation experiments on NMR JNM-ECS400 (JEOL) spectrometer (Akishima, Japan). The echo peak 1800 intensity was recorded at 24 different values of the pulse from 0.005 s to 0.12 s with interval of 0.005 s. Data from T2 relaxometry were fitted to a mono-exponential curve: Signal=B×exp(−TE/T2), where TE is the echo time. Hydrogen atoms from three methylene groups located at side chain of polycations were selected for analysis of PIC nanoparticles.

Inductively coupled plasma mass spectroscopic (ICP-MS) analysis. PIC nanoparticle in 10 mM pH 7.4 PB containing 150 mM NaCl were separated by ultracentrifugation using Desktop Ultracentrifuge Optima MAX-XP (Beckman Coulter) at speeds of 120,000 RPM for 4 h. Separation efficacy was monitored by Cy5 fluorescence of solution. The separated samples were digested in concentrated HNO₃ at 150° C. in acid & alkali resistant fume hood and then redissolved in 1% HNO₃, followed by analysis of sodium content using ICP-MS 7700x (Agilent). Standard calibration curve was obtained from 0.1, 1, 10, 100 ppm NaCl solutions.

Protein binding and macrophage uptake. In vitro protein binding was analysed through fluorescence correlation spectroscopy (FCS) using Zeiss LSM 880 confocal laser-scanning microscope equipped with 40× C-Apochromat water-immersion objective lens (Zeiss, N.A. 1.2) and isothermal titration calorimetry (MicroCal PEAQITC, Malvern). For FCS, briefly, 2 L Cy5-labelled PIC nanoparticles (1 mg mL⁻¹) were added into 200 L PB (10 mM pH 7.4) or 200 L 100% FBS in μ-slide 8 well (Ibidi GmbH, Germany). After 10 min, FCS measurements were performed with He—Ne laser excitation at 647 nm. Ten independent repetitions of autocorrelation functions were collected and analysed in Zeiss ConfoCor systems. For ITC, heat generation was recorded by titrating 20% FBS in phosphate buffered saline into 0.04 mg mL⁻¹ PIC nanoparticles at stir speed of 750 rpm. The titration method made a single 0.4 L injection, followed by 18 injections of 2 L FBS with injection spacing of 150 s and injection duration of 4.0 s. For the sake of simplicity, the molar concentration of FBS was calculated using the albumin molecular weight of 66 KDa (given the major proteins in FBS are albumin) and average protein concentration of 38 g L⁻¹. We used the total polymer concentration as the molar concentration of samples. ITC data were fit to one-site binding model to obtain thermodynamic parameters-enthalpy (ΔH), entropy (ΔS), free energy (ΔG)-and binding affinity (KD), and stoichiometry (N). For evaluating macrophage uptake, RAW264.7 cells were seeded in 96-well plates at a density of 3×10⁴ cells/well and incubated overnight, followed by addition of Cy5-labelled polyion complex nanoparticles at final concentration of 0.1 mg mL⁻¹. After 2 h incubation, the medium was removed and 100 L passive lysis buffer was added for another 1 h incubation. The fluorescence was recorded by microplate reader (TECAN, Infinite M1000 PRO) with a filter set of Ex/Em=649/670 nm.

Intravital real-time confocal laser scanning microscopy (IVRTCLSM) observation and analysis. AIR confocal laser scanning microscopic system attached to an upright ECLIPSE FN1 equipped with a CFI Plan Apo λ 20× objective (Nikon Corp., Tokyo, Japan) was used for intravital observation. The pinhole diameter was adjusted to result in a 10 m optical slice. To determine the longevity and FRET signal in blood, ear lobe dermis was fixed beneath a coverslip with immersion oil. Cy5-labelled or Cy3/Cy5-co-labelled PIC nanoparticles (200 L, 0.5 mg mL⁻¹) were intravenously administered into BALB/c mice 10 s after the start of fluorescence recording. Cy5 was excited by 640-nm diode laser with band-pass emission filter of 700/75 nm. Relative fluorescence intensity (%) was calculated for the regions of interest (ROIs) defined over the vein as follows. Fluorescence intensity (%)=I/Iₘₐₓ×100%, where I and Iₘₐₓ represent background-subtracted intensity of ROIs at indicated time point and background-subtracted maximum intensity of ROIs, respectively. FRET measurements were conducted with excitation at 561 nm with spectral emission range of 569-712 nm at 5 nm spectral resolution. The spectrally unmixed images (Cy3 and Cy5) were then analysed to identify FRET signal for ROIs as follows. FRET signal=(area under the curve of the Cy5 spectrum)/(area under the curve of the Cy3 spectrum). All image analysis was performed using NIS Elements software (Nikon).

To observe the interaction with liver, an abdominal midline skin incision was carefully made to allow the exposure of liver lobe. The exposed liver lobe was fixed beneath a coverslip and 200 L Cy5-labelled PIC nanoparticles (0.5 mg mL⁻¹) were intravenously administered into BALB/c mice 10 s after the start of the start of Cy5 fluorescence recording. For bile duct imaging, at indicated time point (9, 24, 48, and 96 h) after intravenous administration, 5-carboxyfluorescein diacetate (CFDA) was intravenously injected at dose of 0.2 mg kg⁻¹. Five minutes later, CFDA and Cy5 fluorescence were recorded. CFDA was excited by 488-nm laser with band-pass emission filter of 520/50 nm. For tracing macrophage, 10 g F4/80 antibody (eFluor 450) was intravenously injected. Five minutes later, F4/80 and Cy5 fluorescence were recorded. F4/80 antibody (eFluor 450) was excited by 403-nm laser with band-pass emission filter of 520/50 nm. All image analysis was performed using NIS Elements software (Nikon).

T_{1/2} of ultra-long circulating PIC nanoparticles. Blood was collected by venipuncture at different time points (24, 48, 72, 96, 120, and 144 h) post intravenous administration (n=4) and Cy5 fluorescence in plasma was quantified by microplate reader. Semilogarithmic plot of relative fluorescence intensity (%) versus time was drawn. Linear regression of plot was applied to obtain the slope −K. Single-phase T1/was calculated as follows. T_{1/2}=ln2/K.

Flow cytometric analysis and immunofluorescence analysis of cryosections. Cy5-labelled PIC nanoparticles (200 L, 0.5 mg mL⁻¹) were intravenously administered into BALB/c mice (n=3 for short-circulating nanoparticle and n=5 for long-circulating nanoparticle). At indicated time points, single-cell suspensions from the harvested liver tissues were generated by passing through a 100 m cell strainer, followed by removal of erythrocyte using ACK lysis buffer. Single-cell suspensions further were incubated with antibody against CD45 and F4/80 according to manufacturer's instructions. Cells were resuspended in PBS containing 1% BSA and DAPI for flow cytometry measurements (Cell Analyzer LSRFortessa X-20, BD Bioscience). Data were analysed with FlowJo software. For immunofluorescence analysis of liver tissue sections, the livers were sectioned into 10 m thick slices with Cryostat CM1950 (Leica), followed by staining F4/80 antibody and imaging with Zeiss LSM 880 confocal laser-scanning microscope. Pearson's correlation coefficient was quantified by Imaris software.

Biodistribution. Cy5-labelled PIC nanoparticles (200 L, 0.5 mg mL⁻¹) were intravenously administered into BALB/c mice (n=5). At predetermined time points, the mice were perfused with saline and sacrificed. Organs were harvested and subjected to fluorescence analysis with in vivo imaging system (IVIS Spectrum SP-BFM-T1, Perkin Elmer). The tissues were further homogenized in passive lysis buffer, followed by analysis of Cy5 fluorescence using microplate reader (TECAN, Infinite M1000 PRO).

Antitumor and antimetastasis efficacy in orthotopic metastatic triple-negative breast cancer. Female BALB/c mice or BALB/c nude mice bearing orthotopic luciferase-tagged 4T1 or 231/LM2 tumors (50-100 mm³) respectively in mammary gland were randomised (n=5). Mice received intravenous administration of ASNase or ASNase@V (4 U at ASNase-equivalent dose) every five days (q5d). Whole-body bioluminescence was monitored by IVIS with intraperitoneal injection of 150 mg kg⁻¹ luciferin. At day 30 for 4T1 and day 60 for 231/LM2, mice were intraperitoneally injected luciferin. Ten minutes later, the excised lungs were subjected to bioluminescence imaging and tumors were weighed. The lungs were further fixed in Bouin's solution for 24 h to count metastatic nodules. In another independent trials (n=6), at the end of treatment, mice were perfused with saline and subsequent 4% PFA. Lungs were harvested and subjected to H&E staining. The images were acquired through Allin-One Fluorescence Microscope (BZ-X810, KEYENCE).

Quantification of asparagine levels. Female BALB/c mice or BALB/c nude mice bearing orthotopic luciferase-tagged 4T1 or 231/LM2 tumors (50-100 mm³) respectively in mammary gland received intravenous administration of ASNase or ASNase@V (4 U at ASNase-equivalent dose) (n=5). At predetermined time points, blood was collected from abdominal aorta and tumors were harvested. Single-cell suspensions of tumor were counted in 0.4% trypan blue and lysed. Blood samples were centrifuged at 4° C. The obtained tumor lysate and plasma were deproteinized using Deproteinizing Sample Preparation Kit-TCA (abcam, ab204708) and 10 kDa spin columns, respectively. Asparagine was quantified by Asparagine Assay Kit (abcam, ab273333) according to the kit protocol.

Quantification of tumor delivery efficiency. Female BALB/c mice or BALB/c nude mice bearing orthotopic luciferase-tagged 4T1 or 231/LM2 tumors (50-100 mm³) respectively in mammary gland received intravenous administration of Cy5-ASNase, Cy5-ASNase@V (4 U at ASNase-equivalent dose) or empty Cy5-labelled vesicle V_{30.3%} (n=5). At predetermined time points, mice were perfused with saline and sacrificed. Tumors were harvested and were further homogenized in passive lysis buffer, followed by analysis of Cy5 fluorescence using microplate reader (TECAN, Infinite M1000 PRO). For fluorescence analysis of cryosections, the tumors were sectioned into 10 m thick slices with Cryostat CM1950 (Leica), followed by imaging with Zeiss LSM 880 confocal laser scanning microscope.

Antitumor efficacy in orthotopic KPC pancreatic cancer. KPC pancreatic cancer was established through orthotopic injection of 5×10⁵ KPC-Luc cells in the tail of pancreas of female C57BL/6J mice. After 6 days, mice bearing KPC tumors were randomised (n=5) and received intravenous administration of ASNase, ASNase@V, PD-1 antibody (aPD-1), or aPD-1 plus ASNase@V (ASNase: 4 U per mouse, aPD-1: 5 mg kg 1 body weight) every five days for 3 injections in total (q5dx3). Whole-body bioluminescence was monitored by IVIS with intraperitoneal injection of 150 mg kg¹ luciferin and survival time was recorded. In another independent trials (n=5), at day 22 posttreatment, mice were sacrificed and the excised tumors were weighed.

Quantification of desmoplasia and anti-PD-1 antibody delivery in KPC tumor. C57BL/6J mice bearing KPC tumors received intravenous injection of saline, ASNase, or ASNase@V (4 U per mouse). Five days later, the tumors were harvested. Immunofluorescence analysis of tumor sections stained with Alexa Fluor® 488 anti-alpha smooth muscle actin antibody or anti-collagen I antibody (secondary antibody: Alexa Fluor® 488 anti-rabbit IgG H&L) were performed. On the other hand, the mice received injection of Cy5-anti-PD-1 antibody. After 12 hours, to visualize the extravasation of Cy5-anti-PD-1 antibody, 0.25 mg fluorescein isothiocyanate-dextran was injected to label blood vessel 10 min before harvesting tumor. Collagen I-positive area, αSMA-positive area, Cy5-aPD-1 positive extravascular area, and average Cy5-aPD-1 fluorescence were quantified Image J software.

### Results

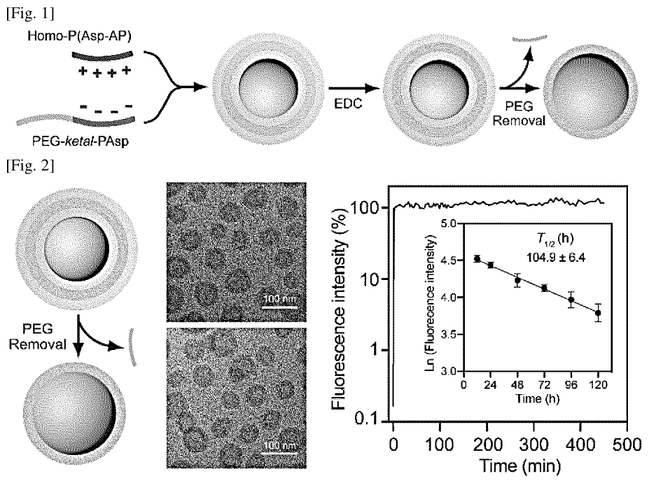

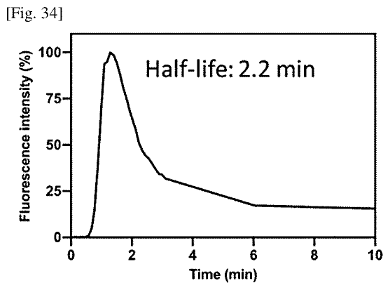

After the polycation and the polyanion synthesized above are mixed to neutralize their charges and obtain a polyion complex vesicle such as a polymersome, the vesicle was subjected to a crosslinking treatment. The vesicle with different crosslinking density was administered to a mouse to evaluate the half-life (T_{1/2}) of the vesicle in the blood circulation by using a fluorescent intensity as an index of the blood vesicle level. As a result, the vesicle with a high crosslinking density exhibited a longer half-life (T_{1/2}). While the half-life (T_{1/2}) of Vₒ% to V_{20.9}% were below 10 minutes, the half-life (T_{1/2}) of V_{30.3}% and V_{93.8}% were 97.2±9.7 hour and 43.1±3.4 hour, respectively (see FIG. 1). To be noted, the half-life (T_{1/2}) of V_{40%} with 10% excess of polycation was 111.1 min (see FIG. 33) and the half-life (T_{1/2}) of V_{40%} with 10% excess of polyanion was 2.2 min (see FIG. 34). Therefore, a neutralization of positive charge and negative charge will increase the half-life of the crosslinked vesicles and micelles.

Then, the surface polyethylene glycol (PEG) was completely removed under the low pH conditions in order to evaluate the effect of PEG surface coating. Although the removal of the surface PEG, the polyion complex vesicle exhibited a very long blood circulation capability and the half-life (T_{1/2}) of the vesicle without PEG coating was 104.9±6.4 hour (see FIG. 2). This result indicates that the PEG is not required to make the vesicle highly stealth, although only PEG is thought to provide a stealth effect to a substance in vivo.

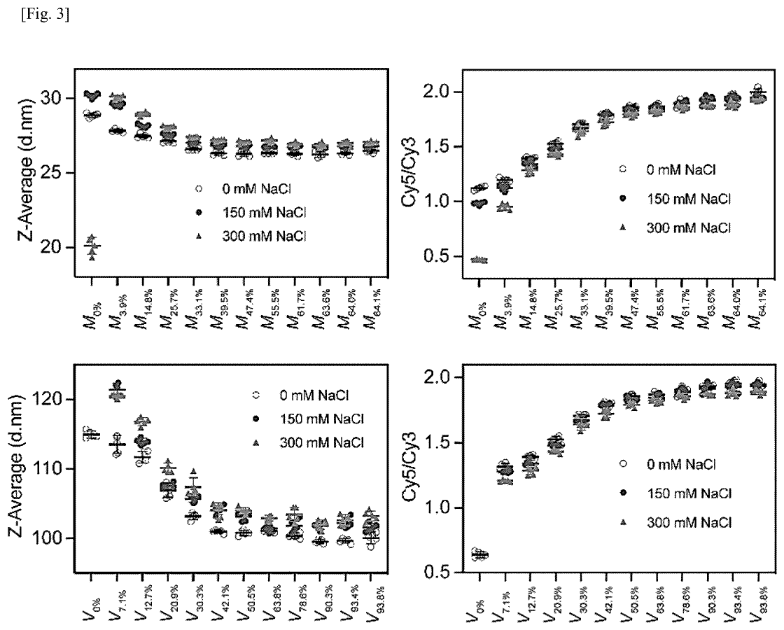

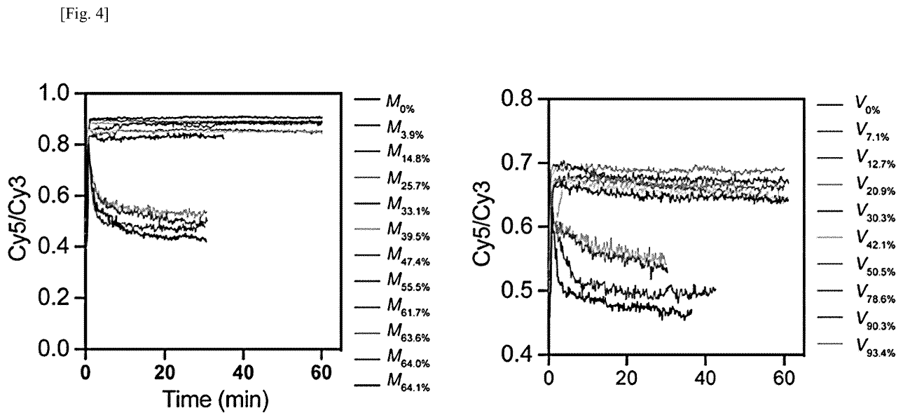

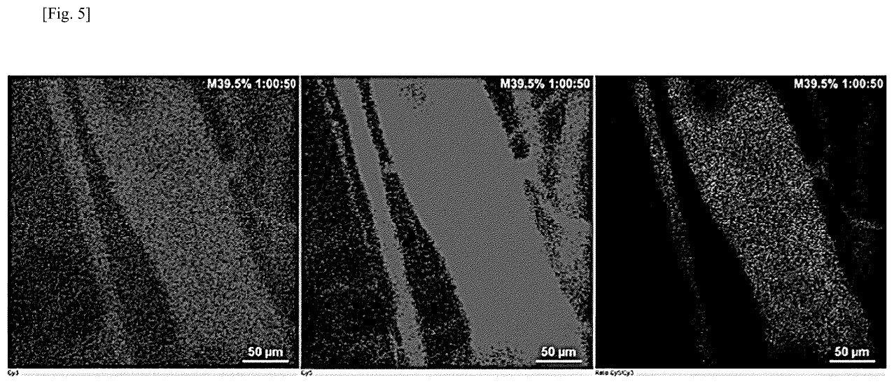

The hydrodynamic diameters of the crosslinked micelles and vesicles were observed. A higher crosslinking density causes a smaller hydrodynamic diameter, which indicates that the polyion complex forms an extensive intermolecular ion-pair network and induces a compact structure. A polyion complex micelle or vesicle consisting of a Cy5-labelled polycation and a Cy3-labelled polyanion were prepared and crosslinked to be subjected to FRET experiment. When Cy5 and Cy3 are positioned within 10 nm in the polyion complex, excitation of Cy3 will result in emission from Cy5. In this FRET experiment, the crosslinking reaction caused increased Cy5 fluorescence/Cy3 fluorescence ratio (Cy5/Cy3) (see FIG. 3), which indicates that the polycation and polyanion are compacted in the crosslinked polyion complex dependent on the crosslinking density. In order to confirm whether or not this compaction is also maintained in a blood circulation, each of the polyion complex micelle or vesicle consisting of a Cy5-labelled polycation and a Cy3-labelled polyanion with various crosslinking density was administered to mice. FIG. 4 shows that micelles with a crosslinking density of 33.1% or more exhibited Cy5 emission for at least 60 minutes (also see FIG. 5 for Cy5 emission and Cy3 emission from M39.5%), while micelles with a crosslinking density below 25.7% exhibited a lower Cy5 emission. FIG. 4 also shows that vesicle with a crosslinking density of 30.3% or more exhibited Cy5 emission for at least 60 minutes, while micelles with a crosslinking density below 20.9% exhibited a lower Cy5 emission. These results indicates that the compaction of the polyion complex by crosslinking can be kept in a blood circulation.

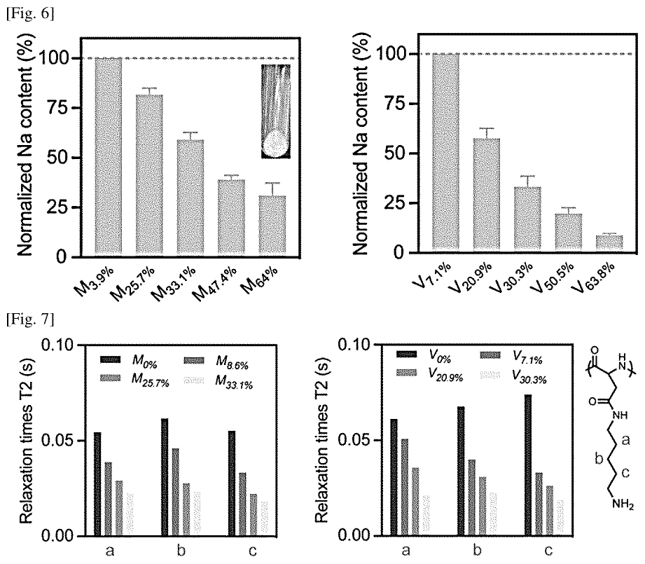

It was thought that almost all of ion pairs form ion pair network and the number of counterions are minimized in the crosslinked polyion complex. In order to confirm whether this is correct or not, a degree of interaction between the crosslinked polyion complex and sodium ion was examined. The crosslinked polyion complex in a phosphate buffer (pH7.4) containing 150 mM NaCl was subjected to ultracentrifugation. After washing the centrifuged crosslinked polyion complex, an amount of sodium ion contained in the centrifuged crosslinked polyion complex was measured. FIG. 6 shows that both of polyion complex micelles and polymersome with a high crosslinking degree avoided interaction with sodium ion. This result shows that the neutralized crosslinked polyion complex has a reduced number of counterions that can interact with an ionic substance such as sodium ion.

Further, the crosslinked polyion complex was subjected to spin-spin relaxation time (T2) measurement. As shows in FIG. 7, the polyion complex with a higher crosslinking degree shows a smaller relaxation time T2 (s), which indicates that the crosslinking treatment reduces the chain flexibility and increases the compaction of the polyion complex. It can be concluded that the ion pair network formed in the polyion complex with minimized number of counterions is stabilized by the crosslinking treatment.

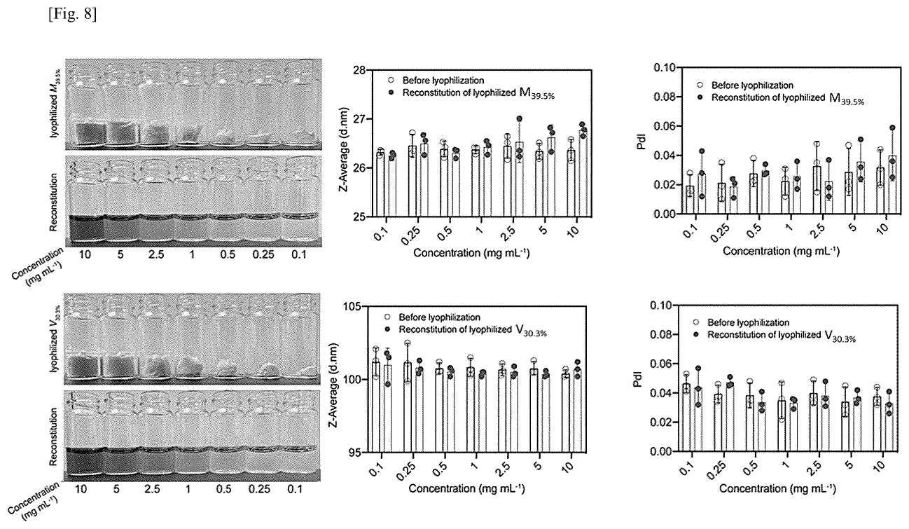

The solubility of the polyion complex with the stabilized ion pair network was examined. The polyion complexed (M_{39.5}% and V_{30.3}%) were each freeze-dried to form a powder composition. Then, the freeze-dried polyion complexed were re-constituted with pure water to examine the solubility of each of the polyion complexed. FIG. 8 shows that the polyion complexes showed a high water-solubility and a superhydrophilicity. Superhydrophilicity of ion-pair network will balance van der Waals attraction and hydrophobic interaction through hydration repulsion.

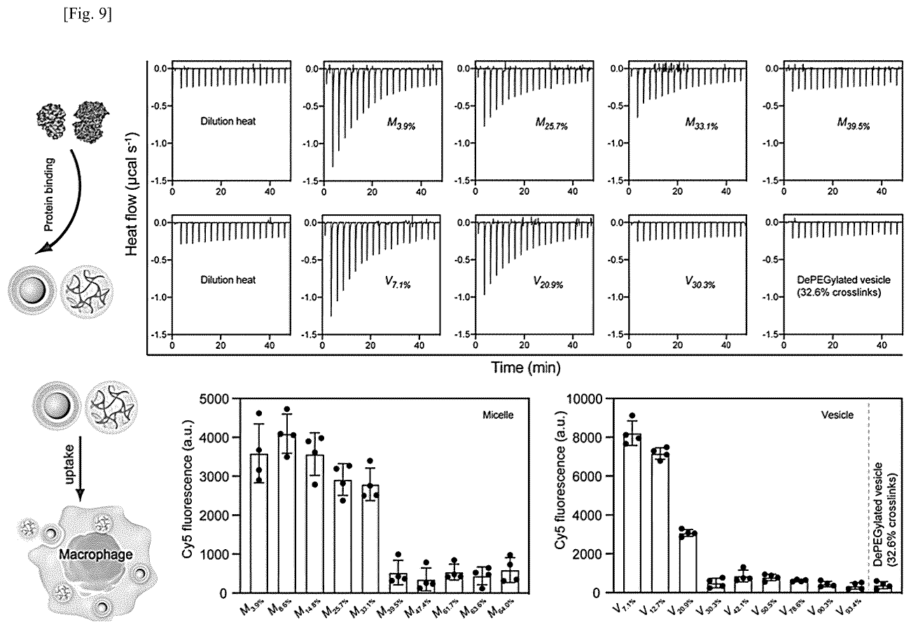

The non-fouling property of the crosslinked polyion complexes was examined by isothermal titration calorimetry (ITC). The uptake of the crosslinked polyion complexes by macrophages involved in innate immunity was also examined. As shown in FIG. 9, the polyion complexes with a higher crosslinking degree escape from binding to serum proteins, which indicates the non-fouling property of the polyion complexes. Further, As shown in FIG. 9, the polyion complexes with a higher crosslinking degree escape from uptake by macrophages, which suggests that the polyion complexes with a higher crosslinking degree can escape from clearance by immunity. Even after a low pH treatment of V_{32.6}% to de-PEGylation of the vesicle, the de-PEGylated vesicle can escape from uptake by macrophages, which further suggests that the PEG coating is unnecessary when the crosslinking degree is sufficiently high.

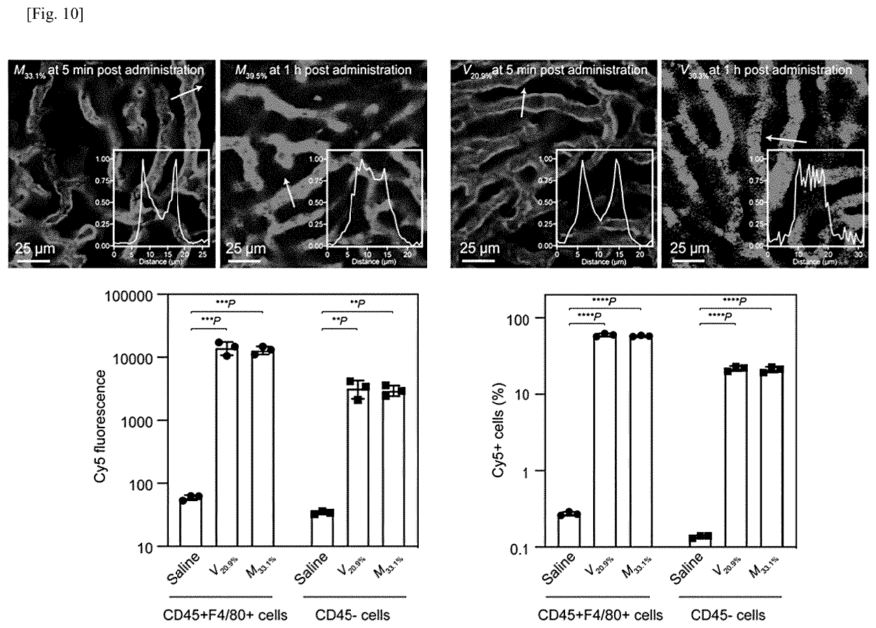

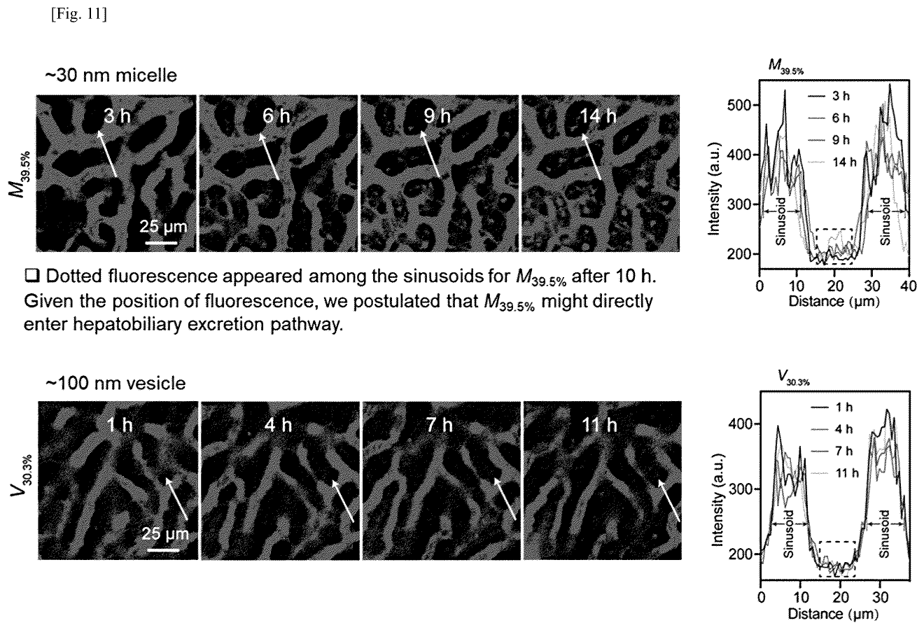

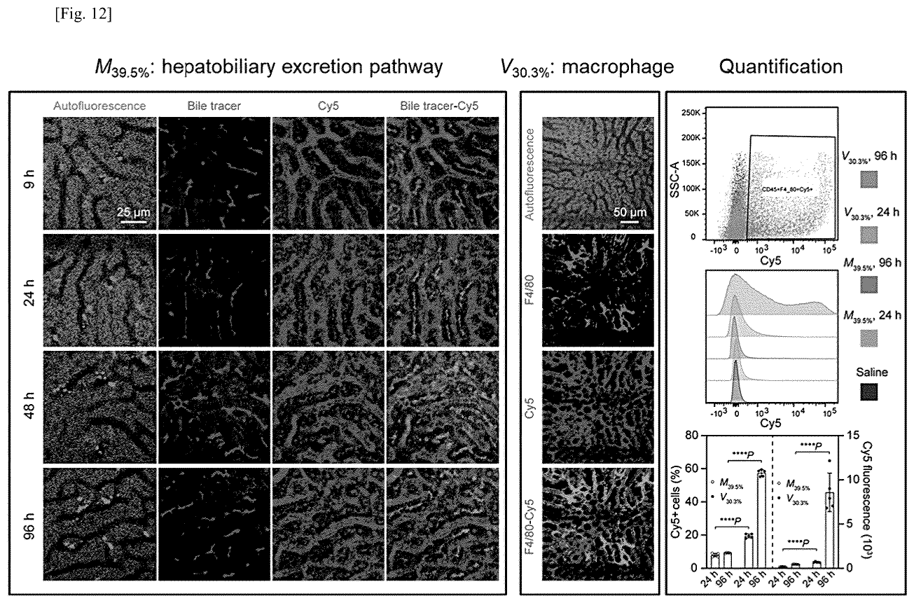

In vivo nano-bio interaction and clearance was examined by intravital microscopy and flow cytometry. As shown in FIG. 10, M33.1% and V20.9% rapidly adhered to sinusoidal wall upon entry into bloodstream compared with M39.5% and V30.3%. Flow cytometric analysis indicated not only macrophage (CD45+F4/80+) but also CD45-populations engaged in uptake of M33.1% and V20.9%. The clearance of long-circulating M39.5% and V30.3% was also examined. As shown in FIG. 11, fluorescence started to appear among the sinusoids for M39.5% after 10 h circulation compared with V30.3%. Furthermore, time-dependent intravital imaging clearly revealed a colocalization of fluorescence with bile canaliculus (FIG. 12). Flow cytometric analysis reconfirmed the distinct metabolic pathways for M39.5% and V30.3% (FIG. 12). V30.3% was found to be slowly captured by CD45+F4/80+ macrophage. M39.5% did not obviously accumulate in CD45+F4/80+ macrophage.

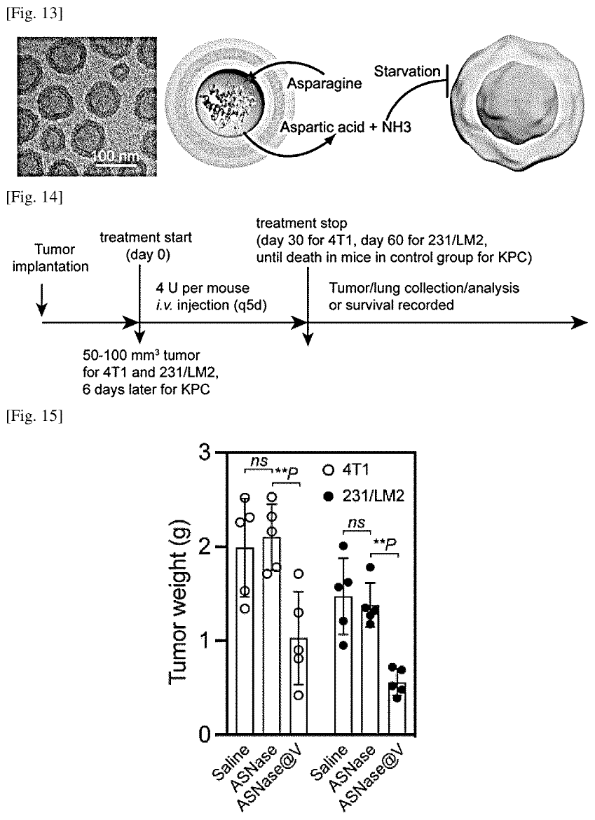

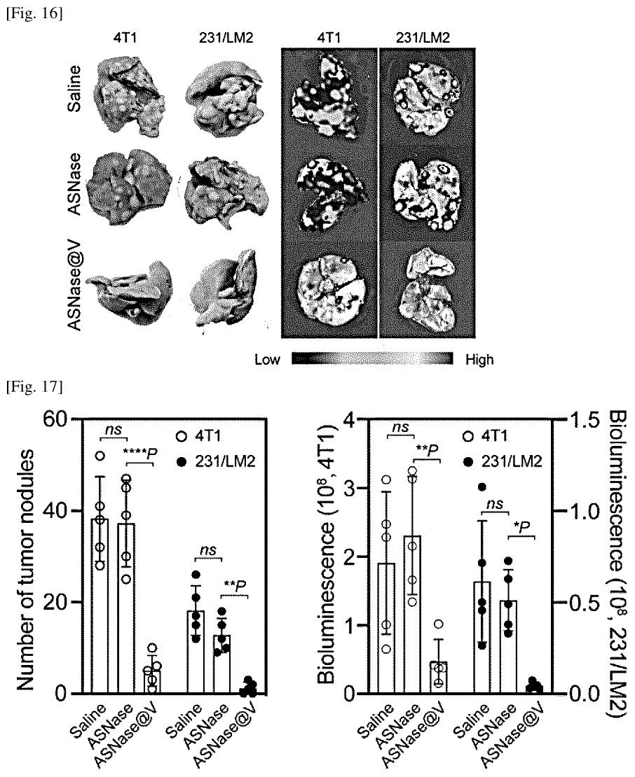

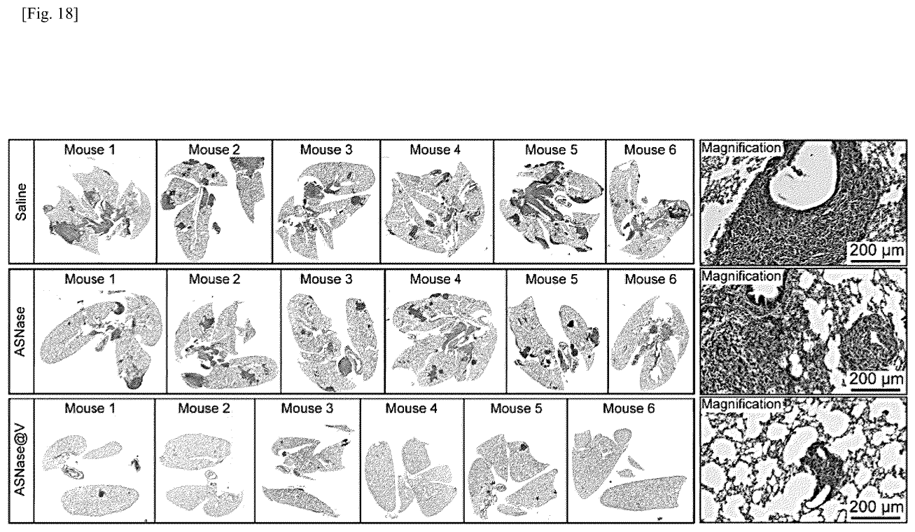

Asparaginase, which can convert asparagine to aspartic acid and NH₃, is used in order to inhibit an asparagine-requiring tumor's growth. In this study, asparaginase was encompassed into the vesicle of the present disclosure to obtain a vesicle encompassing asparaginase (see FIG. 13). Water soluble molecules can penetrate the hydrophilic polyion complex layer in the vesicle, and therefore, asparagine can enter into the vesicle from the outside of the vesicle and aspartic acid and NH₃ can be released from the vesicle. As shown in FIG. 14, the vesicle encompassing asparaginase (˜31.2% crosslinking density) was administered to tumor-bearing mice to examine its anti-tumor effect. The tumor-bearing mice were obtained by grafting tumor (4T1 or 231/LM2) to mammary gland of each of mice. Mice having tumor with 50-100 mm³ in its volume were intravenously (q5d) administered to with the vesicle at a dose of 4 U per mouse. 30 days after the treatment, the mice were analyzed. Weight of the tumor 30 days after the treatment were measured. The vesicle encompassing asparaginase (ASNase@V) inhibits the tumor weight in 4T1 tumor-bearing mice and 231/LM2 tumor-bearing mice (FIG. 15). The lung of each mouse was observed under the fluorescence microscope. FIGS. 16 and 17 show that the vesicle encompassing asparaginase (ASNase@V) prevent metastasis of the grafted tumor. FIG. 18 shows reduction in size and number of metastatic foci through hematoxylin and eosin (H&E) staining after treatment with ASNase@V.

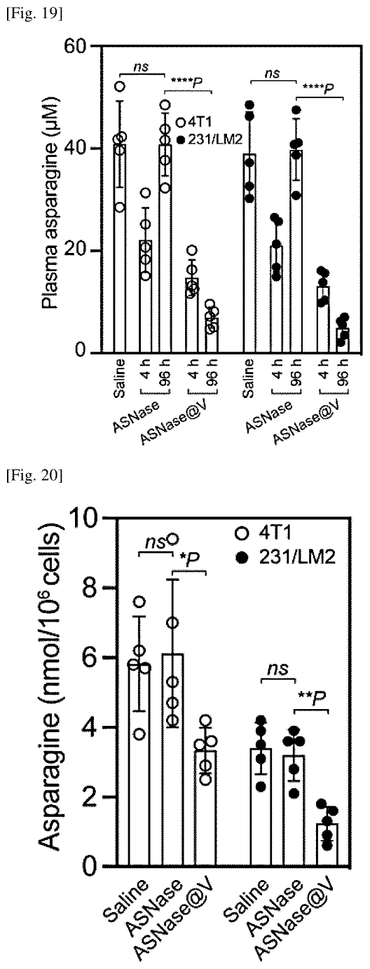

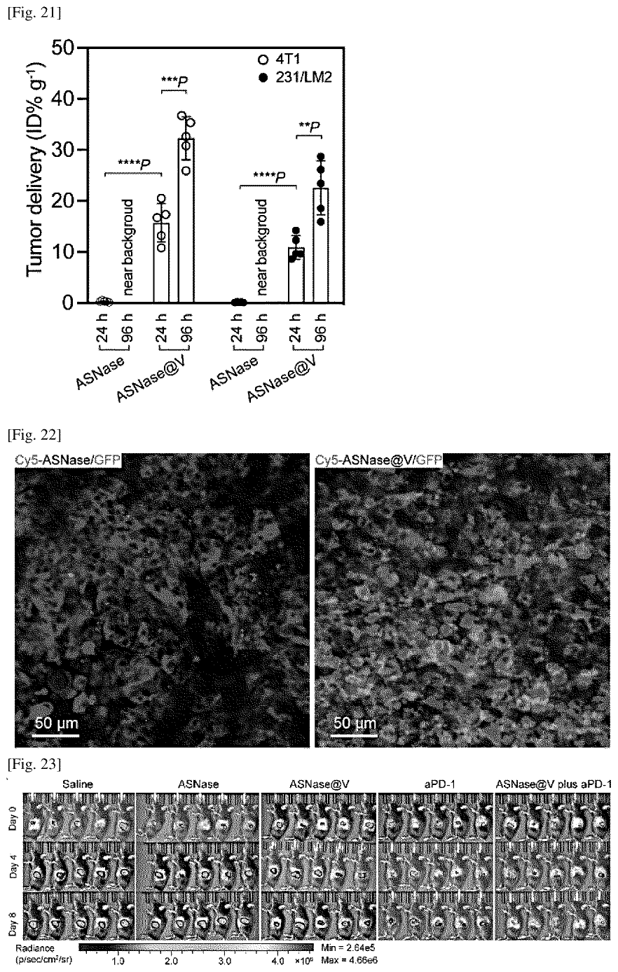

Plasma asparagine level and intratumor asparagine level were measured. The vesicle encompassing asparaginase (ASNase@V) successfully decrease the plasma asparagine level and intratumor asparagine level (FIGS. 19 and 20). Further, Cy5-labelled asparaginase delivery to tumors was observed in BALB/c nude mice bearing orthotopic luciferase-tagged 4T1 or 231/LM2 tumors (50-100 mm3) respectively in mammary gland. The vesicle encompassing asparaginase (ASNase@V) successfully delivered asparaginase to tumors (see FIGS. 21 and 22). The tumor cells were stably transfected with GFP.

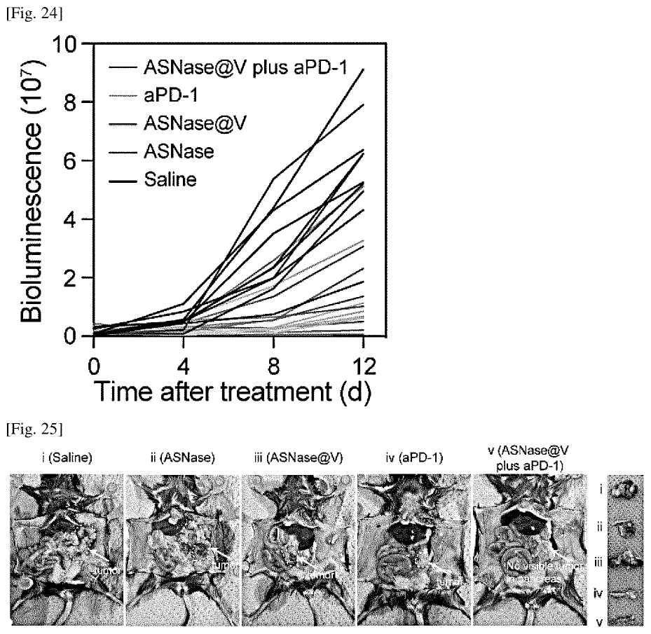

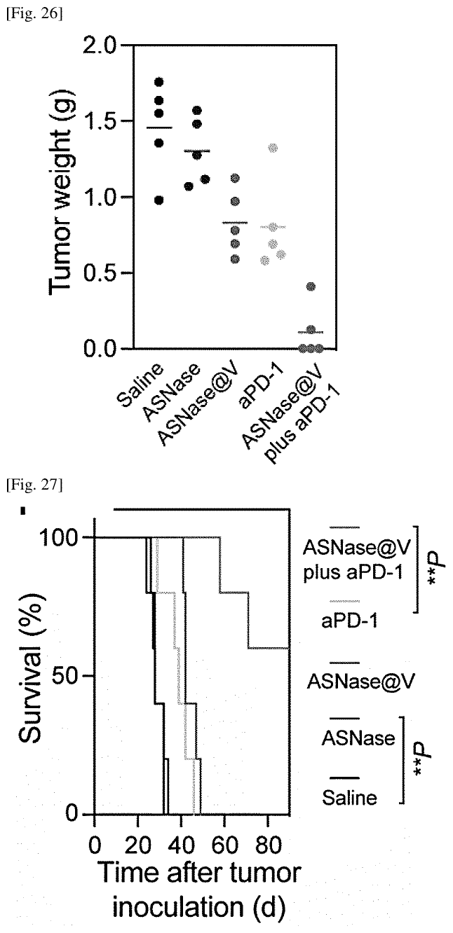

The vesicle encompassing asparaginase (ASNase@V) was administered to pancreatic tumor-bearing mice in combination with an immune checkpoint inhibitor (anti-PD-1 antibody). The vesicle encompassing asparaginase (ASNase@V) successfully inhibit tumor growth in a similar level to the anti-PD-1 antibody, while the combination therapy of the ASNase@V and the anti-PD-1 antibody synergistically and dramatically decreases the tumor weight (FIGS. 23 to 26). The survival of the pancreatic tumor-bearing mice was observed. The asparaginase treated mice were died about 30 days after tumor inoculation, while the ASNase@V clearly improve the survival (see FIG. 27). The combination therapy of the ASNase@V and the anti-PD-1 antibody synergistically and dramatically improved the survival (see FIG. 27).

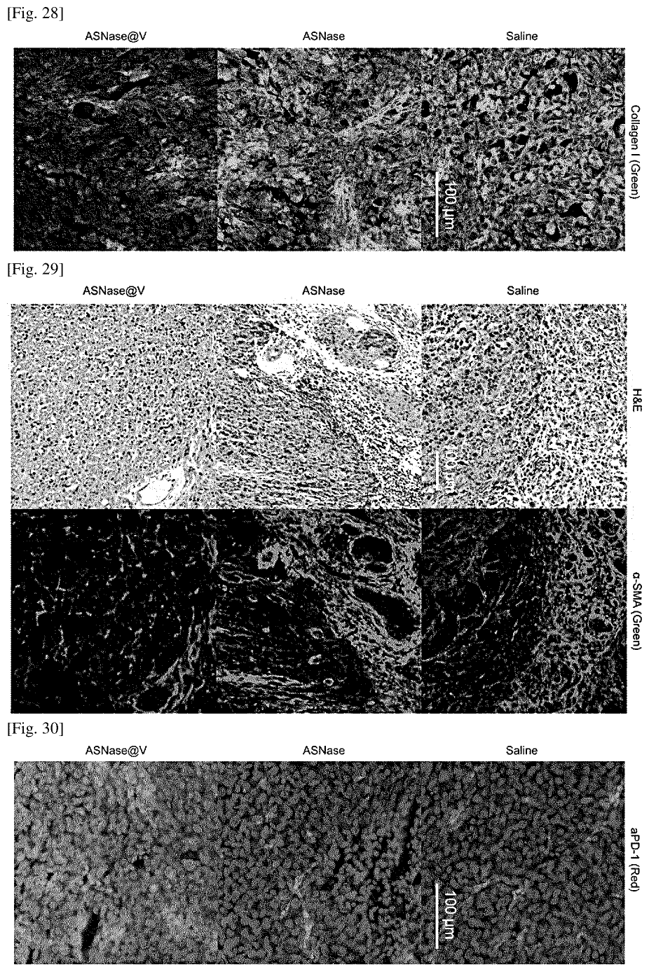

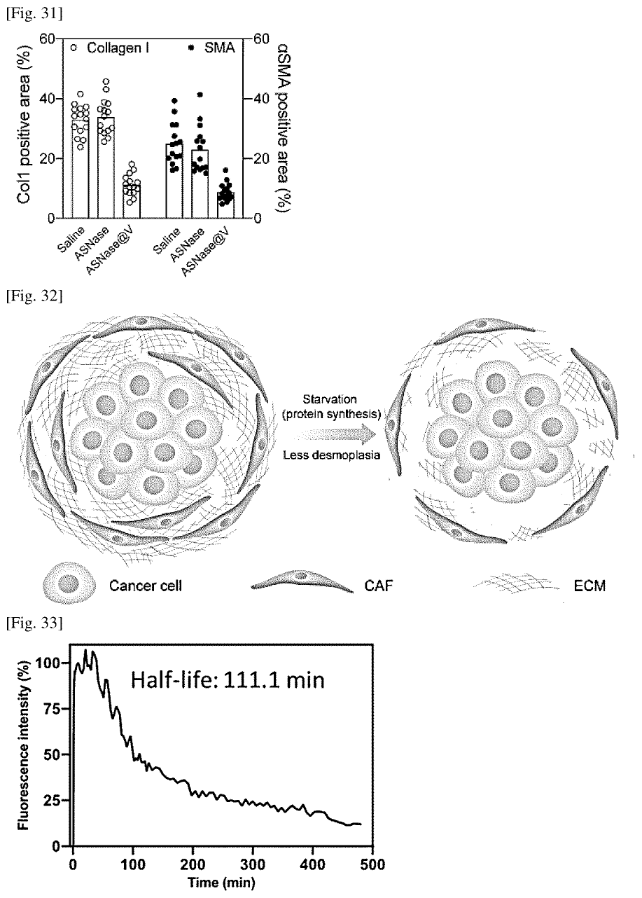

A starvation will prevent desmoplastic reactions such as proliferation of alpha-smooth muscle actin-positive fibroblasts, and deposition of extracellular matrix components in a tumor environment. If the ASNase@V successfully induce a starvation in a tumor, the tumor will have reduced desmoplastic reactions (see FIG. 32). Therefore, the effect of the ASNase@V on the desmoplastic reactions was observed. As shown in FIGS. 28, 29, and 31, the ASNase@V successfully prevent the deposition of collagen I and the proliferation of alpha-smooth muscle actin-positive fibroblasts. As a result, anti-PD-1 antibody (aPD-1) permeated into the tumor tissue observed (see FIG. 30).

High surface energy of synthetic nanomaterials restricts their biomedical applications substantially. To date, it remains insurmountable to construct stealth nanomaterials without steric repulsion. Here we now report an unprecedented steric repulsion-independent bio-stealth technology based on cooperative supramolecular interactions (ion-pair network). Starting from model polyion complex nanoparticle, we elucidate that the cooperative ion-pair network sheath features both superhydrophilicity and minimal unbalanced binding sites. Impressively, fine-tuning the stability of ion-pair network sheath improves circulation half-life by 5500-fold (>100 hours). With minimal interference of nonspecific interaction, for the first time, these model super-stealth nanoparticles credibly define size threshold of targeting liver parenchyma and reveal limited influence of individually tailoring size. Eventually, super-stealth vesicular nanoreactor capacitates asparaginase for therapeutic effect in metastatic breast cancer and pancreatic cancer through strikingly improving bioavailability. Desmoplasia alleviation from severe asparagine starvation further enhances the delivery of anti-PD-1 antibody, leading to synergistic starvation-immunotherapy. Our disruptive observations may capture the importance of consolidating hierarchical biostructures in nature and shift the paradigm for design of nonfouling (stealth) materials from molecularism to structural holism.

High surface energy of nanomaterials driving notorious undesired interactions restricts their biomedical applications substantially¹². Surface hydration plays key roles to lower interfacial energy, resisting interactions at the nano-bio interfaces^{1,3}. However, hydration alone always fails to effectuate stealth properties. For example, liposomes and silica nanoparticles have strongly hydrophilic surface because of zwitterion and hydroxyl groups, whereas they are still prone to absorb proteins and be rapidly cleared by reticuloendothelial system (RES). In other words, non-negligible interfacial energy (or, more precisely, unbalanced binding sites) still exists on wellhydrated surface to drive nano-bio interaction. To dissolve this issue, appending flexible hydrophilic polymer (e.g., PEG, polyzwitterion) has prevailed as the principal conceptual approach (namely steric repulsion) to shield the unbalanced binding sites by forming entropic barrier⁴⁻⁸. Notably, steric repulsion is susceptible to multiple factors. First of all, sufficient polymer coverage is a prerequisite to effective shielding. Despite some attempts have been made to understand the effects of polymer density/thickness and chain topological structures/conformation on steric repulsion, improvements remain limited⁹⁻¹⁵. For example, two-layer PEG (that is, dense inner layer for adequately shielding the core and more dynamic outer layer for balancing entropic cost), maximally extended circulation half-life of PLGA nanoparticles to around 10 h¹⁵. Furthermore, in most cases, modification of polymers is semi-empirically or purely logically applied without quantification. Most of reported hydrophilic polymermodified nanomaterials still showed a short distribution half-life (T_{1/2α})^{7,8}, signifying a rapid nano-bio interaction. Hence, it is imperative and reasonable to develop conceptually distinct stealth technology beyond our unshakable belief of steric repulsion.

Boldly thinking outside the box, another more straightforward way to minimize the interfacial energy is radically abolishing unbalanced binding sites instead of shielding by entropic barrier. To date, it is an insurmountable task owing to the intrinsic high surface energy of nanomaterials. Here we reported this unprecedented approach to construct the low energy surface of nanomaterials by cooperative supramolecular interactions.

The present invention provides polyion complex (PIC) nanoparticles composed of polycations and polyanions naturally integrating an extensive network of intermolecular interactions, mainly ion-pairing. Surprisingly, the inventors found that the steric repulsion of PEG is not required to stealth effect. Instead, PIC nanoparticles circulate over an ultra-long period solely because of stabilization of ion-pair network sheath by crosslinks. It represents a conceptually distinct stealth technology by fabricating stable surface at the sacrifice of entropy, whereas entropic stabilization from molecular flexibility is precisely the core of steric repulsion (Supplementary Movie 1 and Supplementary FIG. 1). This ion-pair network sheath features not only superhydrophilicity by ionic solvation forming hydration barrier but also stable intermolecular interactions minimizing unbalanced binding sites. First, ionic hydration barrier is stronger than that formed by hydrogen bonding (e.g., hydration of PEG). Second, crosslinking reduces chain flexibility and increases chain compaction, enabling positive and negative charges to seamlessly interact with each other to frame ion-pair network. This cooperative ion-pair network has minimal unbalanced charges (counterions) and dipole moments to drive electrostatic binding. The results validated the super-stealth effect with an improvement of monophasic T_{1/2}>5500-fold (from 1 min to 97.2-121.5 h) through fine-tuning the stability of ion-pair network for 30 nm PIC micelle and 100 nm PIC vesicle. Based on these model super-stealth nanoparticles, we credibly unraveled size threshold of targeting liver parenchyma with minimal interference of non-specific interaction. The extremely slow distribution of 30 nm micelle further revealed limited influence of individually tailoring size on targeting liver parenchyma. Eventually, asparaginase-loaded PIC vesicle as super-stealth therapeutic nanoreactor exhibited excellent bioavailability with improved efficacy of starvation therapy in metastatic breast cancer and pancreatic cancer. The results raise the possibility of sensitizing solid tumors to asparagine depletion, which thus far was limited to treat only acute lymphoblastic leukemia (ALL) with asparagine auxotrophy. More interestingly, severe asparagine starvation restricting protein synthesis could alleviate desmoplastic reaction that notoriously counteracts therapeutics delivery in pancreatic cancer. In consequence, combined with anti-PD-1 antibody led to a synergistic starvation-immunotherapy and extended survival in KPC mice. Our disruptive observations may capture the importance of consolidating hierarchical biostructures with much of their surface surrounded by zwitterions or opposite charges and shift the paradigm for design of nonfouling (stealth) materials from molecularism to structural holism.

The documents and patents are hereby incorporated by reference in its entirety.

### REFERENCES

1. Nel, A. E., et al. Understanding biophysicochemical interactions at the nano-bio interface. Nat. Mater. 8, 543-557 (2009).
2. Monopoli, M. P., Aberg, C., Salvati, A. & Dawson, K. A. Biomolecular coronas provide the biological identity of nanosized materials. Nat. Nanotechnol. 7, 779-786 (2012).
3. Chen, S., Li, L., Zhao, C. & Zheng, J. Surface hydration: Principles and applications toward low-fouling/nonfouling biomaterials. Polymer 51, 5283-5293 (2010).
4. McPherson, T. B., Lee, S. J. & Park, K. Analysis of the prevention of protein adsorption by steric repulsion theory. ACS Symp. Ser. 602, 395-404 (1995).
5. Otsuka, H., Nagasaki, Y. & Kataoka, K. PEGylated nanoparticles for biological and pharmaceutical applications. Adv. Drug Deliv. Rev. 55, 403-419 (2003).
6. Keefe, A. J. & Jiang, S. Poly(zwitterionic)protein conjugates offer increased stability without sacrificing binding affinity or bioactivity. Nat. Chem. 4, 59-63 (2011).
7. Lu, Y., et al. Micelles with ultralow critical micelle concentration as carriers for drug delivery. Nat. Biomed. Eng. 2, 318-325 (2018).
8. Chen, S., et al. Enhanced tumour penetration and prolonged circulation in blood of polyzwitterion-drug conjugates with cell-membrane affinity. Nat. Biomed. Eng. 5, 1019-1037 (2021).
9. Walkey, C. D., Olsen, J. B., Guo, H., Emili, A. & Chan, W. C. Nanoparticle size and surface chemistry determine serum protein adsorption and macrophage uptake. J. Am. Chem. Soc. 134, 2139-2147 (2012).
10. Bertrand, N., et al. Mechanistic understanding of in vivo protein corona formation on polymeric nanoparticles and impact on pharmacokinetics. Nat. Commun. 8, 777 (2017).
11. Fox, M. E., Szoka, F. C. & Frechet, J. M. J. Soluble polymer carriers for the treatment of cancer: the importance of molecular architecture. Acc. Chem. Res. 42, 1141-1151 (2009).
12. Perry, J. L., et al. PEGylated PRINT nanoparticles: the impact of PEG density on protein binding, macrophage association, biodistribution, and pharmacokinetics. Nano Lett. 12, 5304-5310 (2012).
13. Yang, Q., et al. Evading immune cell uptake and clearance requires PEG grafting at densities substantially exceeding the minimum for brush conformation. Mol. Pharm. 11, 1250-1258 (2014).
14. Tockary, T. A., et al. Tethered PEG crowdedness determining shape and blood circulation profile of polyplex micelle gene carriers. Macromolecules 46, 6585-6592 (2013).
15. Zhou, H., et al. Dense and dynamic polyethylene glycol shells cloak nanoparticles from uptake by liver endothelial cells for long blood circulation. ACS Nano 12, 10130-10141 (2018).
16. Koide, A., et al. Semipermeable polymer vesicle (PICsome) self-assembled in aqueous medium from a pair of oppositely charged block copolymers: physiologically stable micro-/nanocontainers of water-soluble macromolecules. J. Am. Chem. Soc. 128, 5988-5989 (2006).
17. Rocker, C., Potzl, M., Zhang, F., Parak, W. J. & Nienhaus, G. U. A quantitative fluorescence study of protein monolayer formation on colloidal nanoparticles. Nat. Nanotechnol. 4, 577-580 (2009).
18. Cedervall, T., et al. Understanding the nanoparticle-protein corona using methods to quantify exchange rates and affinities of proteins for nanoparticles. Proc. Natl. Acad. Sci. USA 104, 2050-2055 (2007).
19. Achazi, K., et al. Understanding the interaction of polyelectrolyte architectures with proteins and biosystems. Angew. Chem. Int. Ed. 60, 3882-3904 (2021).
20. Klotz, I. M. Ligand-receptor complexes: origin and development of the concept. J. Biol. Chem. 279, 1-12 (2004).
21. Abdelwahed, W., Degobert, G., Stainmesse, S. & Fessi, H. Freeze-drying of nanoparticles: formulation, process and storage considerations. Adv. Drug Deliv. Rev. 58, 1688-1713 (2006).
22. Kataoka, K., Tsuruta, T., Akaike, T. & Sakurai, Y. Biomedical behavior of synthetic polyion complexes toward blood platelets. Makromol. Chem. 181, 1363-1373 (1980).
23. Wada, A. & Nakamura, H. Nature of the charge distribution in proteins. Nature 293, 757-758 (1981).
24. Nakamura, H. Roles of electrostatic interaction in proteins. Q. Rev. Biophysics 29, 1-90 (1996).
25. Lai, S. K., Wang, Y. Y. & Hanes, J. Mucus-penetrating nanoparticles for drug and gene delivery to mucosal tissues. Adv. Drug Deliv. Rev. 61, 158-171 (2009).
26. Kumar, R. & Fredrickson, G. H. Theory of polyzwitterion conformations. J. Chem. Phys. 131, 104901 (2009).
27. Delgado, J. D. & Schlenoff, J. B. Static and dynamic solution behavior of a polyzwitterion using a hofmeister salt series. Macromolecules 50, 4454-4464 (2017).
28. Huang, C. & Mason, J. T. Geometric packing constraints in egg phosphatidylcholine vesicles. Proc. Natl. Acad. Sci. USA 75, 308-310 (1978).
29. Allen, C., et al. Controlling the physical behavior and biological performance of liposome formulations through use of surface grafted poly (ethylene glycol). Biosci. Rep. 22, 225-250 (2002).
30. Tirosh, O., Barenholz, Y., Katzhendler, J. & Priev, A. Hydration of polyethylene glycol-grafted liposomes. Biophys. J. 74, 1371-1379 (1998).
31. Garbuzenko, O., Barenholz, Y. & Priev, A. Effect of grafted PEG on liposome size and on compressibility and packing of lipid bilayer. Chem. Phys. Lipids 135, 117-129 (2005).
32. Arnold, K., Herrmann, A., Pratsch, L. & Gawrisch, K. The dielectric properties of aqueous solutions of poly(ethylene glycol) and their influence on membrane structure. Biochim. Biophys. Acta 815, 515-518 (1985).
33. Sorensen, K. K., Simon-Santamaria, J., McCuskey, R. S. & Smedsrod, B. Liver sinusoidal endothelial cells. Compr. Physiol. 5, 1751-1774 (2015).
34. Dirisala, A., et al. Transient stealth coating of liver sinusoidal wall by anchoring two-armed PEG for retargeting nanomedicines. Sci. Adv. 6, eabb8133 (2020).
35. Li, J., Anraku, Y. & Kataoka, K. Self-boosting catalytic nanoreactors integrated with triggerable crosslinking membrane networks for initiation of immunogenic cell death by pyroptosis. Angew. Chem. Int. Ed. 59, 13526-13530 (2020).
36. Anraku, Y., et al. Systemically injectable enzyme-loaded polyion complex vesicles as in vivo nanoreactors functioning in tumors. Angew. Chem. Int. Ed. 55, 560-565 (2016).
37. Lohr, M., et al. Transforming growth factor-β1 induces desmoplasia in an experimental model of human pancreatic carcinoma. Cancer Res. 61, 550-555 (2001).
38. Krall, A. S., Xu, S., Graeber, T. G., Braas, D. & Christofk, H. R. Asparagine promotes cancer cell proliferation through use as an amino acid exchange factor. Nat. Commun. 7, 11457 (2016).
39. Rassolian, M., Chass, G. A., Setiadi, D. H. & Csizmadia, I. G. Asparagine-ab initio structural analyses. J. Mol. Struct.: THEOCHEM 666-667, 273-278 (2003).
40. Knott, S. R. V., et al. Asparagine bioavailability governs metastasis in a model of breast cancer. Nature 554, 378-381 (2018).
41. Hammel, P., et al. Erythrocyte-encapsulated asparaginase (eryaspase) combined with chemotherapy in second-line treatment of advanced pancreatic cancer: An open-label, randomized Phase IIb trial. Eur. J. Cancer 124, 91-101 (2020).

1. A polyion complex, comprising polycation and polyanion,
wherein the polycation has a positive charge, and comprises (i) a cationic polymer or a block copolymer comprising a cationic polymer block and optionally (ii) an uncharged hydrophilic polymer block,
wherein the polyanion has a negative charge, and comprises (iii) an anionic polymer or a block copolymer comprising an anionic polymer block and optionally (iv) an uncharged hydrophilic polymer block,
wherein the polycation and polyanion interact with each other to form the polyion complex,
wherein the positive charge and the negative charge are neutralized in the polyion complex, and the ratio of the positive charge to the negative charge ranges between 0.95 and 1.05,
wherein the polycation and polyanion are stabilized by crosslinking in the polyion complex and have a crosslinking density, and
wherein the polyion complex has a half-life (T_{1/2}) over 24 hours in a blood or serum of the subject.

2. The polyion complex of claim 1, forming a polyion complex micelle.

3. The polyion complex of claim 1, forming a polyion complex polymersome.

4. The polyion complex of claim 2, wherein the crosslinking density is over 30%.

5. The polyion complex of claim 2, wherein the crosslinking density is over 40%.

6. The polyion complex of claim 3, wherein the crosslinking density is over 30%.

7. The polyion complex of claim 3, wherein the crosslinking density is over 40%.

8. The polyion complex of claim 2, wherein the sodium content in the polyion complex is 60% or less of that in a polyion complex micelle having 3.9% crosslinking density after four-hour centrifugation in 10 mM phosphate buffer (pH7.4) containing 150 mM NaCl.

9. The polyion complex of claim 2, wherein the sodium content in the polyion complex is 50% or less of that in a polyion complex micelle having 3.9% crosslinking density after four-hour centrifugation in 10 mM phosphate buffer (pH7.4) containing 150 mM NaCl.

10. The polyion complex of claim 3, wherein the sodium content in the polyion complex is 60% or less of that in a polyion complex polymersome having 7.1% crosslinking density after four-hour centrifugation in 10 mM phosphate buffer (pH7.4) containing 150 mM NaCl.

11. The polyion complex of claim 3, wherein the sodium content in the polyion complex is 50% or less of that in a polyion complex polymersome having 7.1% crosslinking density after four-hour centrifugation in 10 mM phosphate buffer (pH7.4) containing 150 mM NaCl.

12. The polyion complex of claim 2, wherein an amount of heat generation as measured by isothermal titration calorimetry after mixing 0.04 mg/mL polyion complex micelle with 20% fetal bovine serum in phosphate-buffered saline ranges between −0.1 and −0.5.

13. The polyion complex of claim 2, wherein an amount of heat generation as measured by isothermal titration calorimetry after mixing 0.04 mg/mL polyion complex micelle with 20% fetal bovine serum in phosphate-buffered saline ranges between −0.1 and −0.4.

14. The polyion complex of claim 3, wherein an amount of heat generation as measured by isothermal titration calorimetry after mixing 0.04 mg/mL polyion complex polymersome with 20% fetal bovine serum in a phosphate-buffered saline ranges between −0.1 and −0.5.

15. The polyion complex of claim 3, wherein an amount of heat generation as measured by isothermal titration calorimetry after mixing 0.04 mg/mL polyion complex polymersome with 20% fetal bovine serum in a phosphate-buffered saline ranges between −0.1 and −0.4.

16. The polyion complex of claim 1, wherein the heat generated at the first titration is 200% or less of the dilution heat by the FBS addition in isothermal titration calorimetry (ITC), preferably (i) wherein the polyion complex is a polyion complex micelle with a crosslinking density over 40% or (ii) wherein the polyion complex is a polyion complex polymersome with a crosslinking density over 30%.

17. A pharmaceutical composition, comprising the polyion complex of claim 1.

18. The pharmaceutical composition of claim 17, encapsulating a therapeutic agent.

19. The pharmaceutical composition of claim 18, wherein the therapeutic agent is asparaginase.

20. The pharmaceutical composition of claim 19, to be administered in combination with another anti-tumor agent.

21. The pharmaceutical composition of claim 20, wherein said another anti-tumor agent is an immune checkpoint inhibitor.
